(19) United States
(12) Patent Application Publication
(10) Pub. No.: US 2007/0265221 A1
(43) Pub. Date: Nov. 15, 2007

(54) Methods to improve creatine kinase metabolism and contractile function in cardiac muscle for the treatment of heart failure

(76) Inventors: Robert G. Weiss, Hunt Valley, MD (US); Hunter Champion, Baltimore, MD (US)

Correspondence Address: WILEY REIN LLP, 1776 K. STREET N.W., WASHINGTON, DC, 20006, US

(21) Appl. No.: 11/797,835

(22) Filed: May 8, 2007

Related U.S. Application Data
(60) Provisional application No. 60/798,886, filed on May 9, 2006.

(51) Int. Cl. A61K 31/70; A61B 5/05

(52) U.S. Cl. 514/44; 600/412

(57) ABSTRACT

The present invention relates to novel treatments of mammalian and human heart failure directed at improving cardiac creatine kinase metabolism, the prime energy reserve of cardiac muscle. The invention also relates to novel treatments using gene transfer vectors to increase myocardial creatine kinase protein expression and/or creatine kinase activity, as well as flux through the creatine kinase reaction and to thereby improve cardiac contractile function and ameliorate remodeling in heart failure. The invention further relates to methods for screening and identifying compounds that increase creatine kinase expression and/or creatine kinase activity as potential pharmaceutical compositions for heart failure therapy.

### CROSS-REFERENCE TO RELATED APPLICATIONS

This application claims the benefit of U.S. provisional application No. 60/798,886, filed May 9, 2006, which is hereby incorporated by reference as though fully set forth herein.

### GOVERNMENT RIGHTS STATEMENT

The United States government may have rights in this invention. This invention was funded (in part) by NIH Grant Numbers HL61912 and HL63030.

### BACKGROUND OF THE INVENTION

1. Field of the Invention

In one aspect, the invention relates in general to novel methods of treating and preventing heart failure. In one aspect, the invention relates to increasing the expression of genes encoding for the isoforms of creatine kinase so as to increase creatine kinase metabolism in the heart and thereby improve cardiac contractile function and limit adverse heart remodeling in heart failure. In another aspect, the invention relates to novel methods of using a vector construct comprising a DNA sequence that codes for creatine kinase to improve creatine kinase metabolism, and/or cardiac contractile function, and/or to favorably alter adverse heart remodeling in patients with or at risk of heart failure. In another aspect, the invention relates to novel methods of identifying pharmacologic agents that cause a detectable increase in creatine kinase expression or activity in cardiac myocytes.

2. Background Art

Heart failure (HF) is typically defined as an inability of the heart to pump sufficient blood to meet the needs of the body at normal filling pressures.

5.2 million people in the United States have HF and there are more than 1 million HF hospitalizations each year with an overall estimated annual cost of $33.2 billion (American Heart Association, Heart and Stroke Statistical Update 2007, Dallas, Tex.).

ATP is the major biochemical form of energy in heart muscle and is required to fuel ongoing normal cardiac contractile function. Although the possibility that inadequate ATP supply could cause HF has been postulated (Ingwall 1993), there has been no direct evidence that this is the case and no metabolic means identified to increase ATP availability in the failing heart (Katz 1998).

There are many reactions in numerous metabolic pathways that contribute to the generation and/or the availability of ATP in the heart. The creatine kinase reaction is one reaction that affects cardiac ATP availability. Creatine kinase is a protein that is expressed in cells. Creatine kinase (CK) is the major energy reserve of the heart, providing ATP cyclically during the cardiac cycle and during periods of increased demand, as it rapidly and reversibly converts creatine phosphate (PCr) and ADP to ATP and creatine (Cr) (Ingwall et al. 1985, Saks 1980, Wallimann 1994). There are three main isoforms of CK; a muscle isoform (CK-M), a brain isoform (CK-B) and a mitochondrial isoform (CK-mito). Abnormalities in cardiac CK metabolism are present in nearly all types of experimental and clinical HF, including a reduction in CK substrates, a switch to fetal CK isoforms, and reduced total CK activity (Nascimben et al. 1996). Despite these findings, and the extraordinary personal and financial toll of human HF, there are no therapies directed at increasing cardiac CK metabolism.

It is not at all clear, however, whether altered cardiac CK energy metabolism causes the contractile dysfunction of HF or is simply one of its many consequences. There is no prior evidence that CK is an appropriate therapeutic target for heart failure therapy and there are, in fact, several lines of evidence that would lead one experienced in the art to expect that reduced CK energy metabolism does not contribute to, or cause, HF. First, transgenic mice lacking major CK genes in the heart (CK knock-out mice for both CK-M and CK-mito) have been created and they do not develop overt HF (Saupe et al. 1998). This suggests that reduced CK metabolism is not a cause of contractile dysfunction or HF since the absence of the gene does not cause HF. In addition, the degree of depletion of the substrates and products of the CK reaction in human heart failure is relatively modest, on the order of 10%-30% (Starling et al. 1998), and is not to a level that would be expected to limit ATP delivery and cardiac function. Finally, chronic supplementation of creatine, a substrate for the CK reaction which is depleted in HF, does not improve contractile function in an animal model of heart failure (Horn et al. 1999). Thus, it is not known or expected by those familiar with this art that increasing CK metabolism represents a treatment for HF that is likely to succeed.

### SUMMARY OF THE INVENTION

The novel methods of the invention address the lack of effective ways to treat and prevent heart failure by influencing the creatine kinase reaction and thereby increasing the supply of energy for cardiac cells. The invention provides novel treatments of mammalian and human heart failure directed at improving cardiac creatine kinase metabolism, the prime energy reserve of cardiac muscle. The methods relate to the use of gene transfer vectors to increase myocardial creatine kinase protein expression and/or creatine kinase activity, as well as flux through the creatine kinase reaction and to thereby improve cardiac contractile function and ameliorate remodeling in heart failure. The method also includes screening and identifying compounds that increase creatine kinase expression and/or creatine kinase activity as potential pharmaceutical compositions for heart failure therapy.

Several aspects of this invention relate to novel methods of using a vector construct to treat or prevent mammalian and human heart failure. Other aspects relate to causing detectable increases of creatine kinase metabolism or improvements in cardiac contractile function or adverse heart remodeling in mammals. Thus, as defined herein, any reference to “mammal” or “mammalian” includes, but is not limited to, “human.” In other aspects of the invention, the vector can include, but is not limited to, a plasmid, phage, transposon, cosmid, chromosome, virus, virion, or any other genetic element, which is capable of replication when associated with the proper control elements and which can function to transfer a gene sequence to a cell. Any of the variety of vectors can be used to perform the steps of the novel methods of this invention related to treating a mammal at risk for or with heart failure, or the novel methods of this invention related to increasing creatine kinase metabolism, and/or increasing cardiac contractile function, and/or improving adverse heart remodeling.

Another aspect of the invention relates to administering the vector construct to a mammalian heart. Any of the methods pertaining to administration can be performed by one or more of the following techniques, including but not limited to, intramuscular, intravenous, intra-arterial, subcutaneous, and intraperitoneal injection, or introduction into cardiac muscle (such as a ventricular or atrial wall) or into the pericardial space, using catheter-based or surgical techniques. In another aspect, the construct can be administered by using a technique to allow for enhanced cardiac transduction. In another aspect, the vector construct can be infused into a blood vessel that perfuses the heart. In yet another aspect, a catheter is inserted into a blood vessel and advanced into a coronary artery that perfuses a portion of the myocardium and the vector is administered through the catheter to the heart.

In another aspect, aortic cross-clamping and whole body cooling can improve significant vector transduction in any of the methods of the invention in which a vector construct is administered.

One aspect of this invention relates to novel methods of using a vector construct having a nucleotide sequence coding for creatine kinase to improve creatine kinase metabolism in a mammal. In one aspect, a vector construct having a nucleotide sequence for a mammalian creatine kinase is provided and administered to a mammal's heart causing a detectable change in creatine kinase metabolism.

In a further aspect, the detectable change comprises and/or can be measured by a change in the ratio of cardiac creatine phosphate to ATP. In another aspect, the change comprises and can be measured by a change in the cardiac concentration of creatine phosphate and/or ATP. In yet another aspect, the change comprises and can be measured by a change in flux through the cardiac creatine kinase reaction.

In a further aspect, the nucleotide sequence can code for a brain isoform of creatine kinase. In another aspect, the nucleotide sequence can code for a muscle isoform of creatine kinase. In yet another aspect, the nucleotide sequence can code for a mitochondrial isoform of creatine kinase. In a further aspect, the nucleotide sequence can code for human isoforms of creatine kinase, including a brain isoform, a muscle isoform, and/or a mitochondrial isoform.

Another aspect of this invention relates to novel methods of using a vector construct having a nucleotide sequence coding for mammalian creatine kinase to improve cardiac contractile function in a mammal. A vector construct having a nucleotide sequence for a mammalian creatine kinase is provided and administered to a mammal's heart causing a detectable increase in cardiac contractile function.

In a further aspect, the detectable increase comprises and can be measured by, but is not limited to, a change in the left or right ventricular ejection fraction and/or an increase in cardiac index.

In another aspect, the nucleotide sequence can code for a brain isoform of creatine kinase. In another aspect, the nucleotide sequence can code for a muscle isoform of creatine kinase. In another aspect, the nucleotide sequence can code for a mitochondrial isoform of creatine kinase. In yet a further aspect, nucleotide sequence can code for human isoforms of creatine kinase, including a brain isoform, a muscle isoform, and/or a mitochondrial isoform.

In another aspect, the increase in contractile function can be measured by any of a number of methods, including, but not limited to, x-ray; x-ray angiography; ultrasound or echocardiography; nuclear medicine ventriculography or scintigraphy; positron emission tomography; magnetic resonance imaging; computed tomography; and/or invasive and non-invasive hemodynamics.

Another aspect of this invention relates to novel methods of using a vector construct having a nucleotide sequence coding for mammalian creatine kinase to improve adverse heart remodeling in a mammal. A vector construct having a nucleotide sequence for a mammalian creatine kinase is provided and administered to a mammal's heart causing an improvement in ventricular size or shape.

In another aspect, the improvement can comprise and/or be measured by, but is not limited to, the mammal's end diastolic volume, end systolic volume, ventricular wall thickness, and/or ventricular mass.

In a further aspect, the improvement in adverse heart remodeling can be measured, for example, by imaging tests, such as x-ray, x-ray angiography, ultrasound or echocardiography, nuclear medicine ventriculography or scintigraphy, positron emission tomography, magnetic resonance imaging, computed tomography, and/or by the use of biomarkers that correlate with remodeling.

In a further aspect, the nucleotide sequence can code for a brain isoform of creatine kinase. In another aspect, the nucleotide sequence can code for a muscle isoform of creatine kinase. In another aspect, the nucleotide sequence can code for a mitochondrial isoform of creatine kinase. In yet a further aspect, the nucleotide sequence can code for human isoforms of creatine kinase, including a brain isoform, a muscle isoform, and/or a mitochondrial iso form.

One aspect of this invention relates to novel methods for identifying a pharmacologic agent or compound that increases cardiac creatine kinase metabolism and/or prevents its decline in HF.

Another aspect of this invention relates to novel methods for identifying pharmacologic agents that increase creatine kinase expression in muscle cells. An aspect of the method of identification includes providing isolated muscle cells in culture, contacting the pharmacologic agent with one or more of the muscle cells, and detecting an increase in creatine kinase expression. In one embodiment, potential pharmacologic agents can be selected from any of a number of test compounds.

In another aspect of the invention, the isolated muscle cells can be transduced with a reporter construct under the control of a creatine kinase promoter.

In yet another aspect, the detection of the increase in creatine kinase expression is by a high-throughput system designed to reflect creatine kinase expression.

In another embodiment of this invention, one or more of the cells can be exposed to an injurious agent or system that mimics characteristics of heart failure (including, but not limited to, a reactive oxygen species generating system such as exposure to hydrogen peroxide). Some of the cells can then be contacted with any of the small molecule test compounds and the CK expression of those cells can be compared to that in cells contacted with inactive (control) pharmacologic agents to identify pharmacologic agents that increase reduced CK expression caused by the injurious agent or system.

In another embodiment of this invention, one or more of the cells can be contacted with any of the small molecule test compounds identified in a library of test compounds before exposure to an injurious agent or system as described above and the CK expression of those cells can be compared to that in cells contacted with inactive (control) agents to identify pharmacologic agents (from the small molecule test compounds) that increase CK expression. Increase as used in this embodiment, is meant to refer to preventing or ameliorating the reduced CK expression that would be expected to result from exposure to the injurious agent or system in the absence of contact with a test compound.

In another embodiment, the muscle cells can be cardiac cells obtained from a mammal with heart failure.

Yet another aspect of this invention is a pharmacologic agent screen for identifying molecules or test compounds that increase CK activity. In one aspect, this method comprises providing isolated and purified creatine kinase, contacting the test compound with the purified creatine kinase, and detecting whether there is an increase in creatine kinase activity.

In another embodiment, the detection of the increase in creatine kinase activity is by a high-throughput system designed to reflect creatine kinase activity.

In another embodiment of this invention, one or more aliquots of the isolated CK isoforms can be exposed to an injurious agent or system that mimics characteristics of heart failure (including, but not limited to, a reactive oxygen species generating system such as exposure to hydrogen peroxide) while other aliquots of isolated CK isoforms are not exposed (control). The exposed and non-exposed CK enzyme can then be contacted with any of the small molecule test compounds identified in a library of test compounds and CK activity compared so as to identify pharmacologic agents that increase reduced CK activity caused by the injurious agent or system.

In yet another embodiment of this invention, one or more aliquots of the isolated CK isoforms can be contacted with any of the small molecule test compounds identified in the library described above before exposure to an injurious agent or system as described above and the CK activity compared to identify pharmacologic agents that prevent or ameliorate the reduced CK activity that would be expected to result from exposure to the injurious agent or system.

Another aspect of this invention relates to novel methods for treating a mammal at risk for or with heart failure using a vector construct having a nucleotide sequence coding for creatine kinase so as to improve creatine kinase metabolism. A vector construct having a nucleotide sequence for a mammalian creatine kinase is provided and administered to a subject's heart causing a detectable change in creatine kinase metabolism.

In a further aspect, the detectable change comprises and can be measured by a change in the ratio of cardiac creatine phosphate to ATP. In another aspect, the change comprises and can be measured by a change in the cardiac concentration of creatine phosphate and/or ATP. In another aspect, the change comprises and can be measured by a change in flux through the cardiac creatine kinase reaction.

In another aspect, the nucleotide sequence can code for a brain isoform of creatine kinase. In another aspect, the nucleotide sequence can code for a muscle isoform of creatine kinase. In another aspect, the nucleotide sequence can code for a mitochondrial isoform of creatine kinase. In a further aspect, the nucleotide sequence can code for human isoforms of creatine kinase, including a brain isoform, a muscle isoform, and/or a mitochondrial isoform.

Another aspect of this invention relates to novel methods for treating a subject at risk for or with heart failure using a vector construct having a nucleotide sequence coding for creatine kinase so as to increase contractile function. A vector construct having a nucleotide sequence for a mammalian creatine kinase is provided and administered to a subject's heart causing a detectable increase in cardiac contractile function.

In a further aspect, the detectable increase comprises and can be measured by, but is not limited to, a change in the left or right ventricular ejection fraction and/or an increase in cardiac index.

In a further aspect, the increase in cardiac contractile function can be measured by a number of methods, including but not limited to, x-ray, x-ray angiography, ultrasound or echocardiography, nuclear medicine ventriculography or scintigraphy, positron emission tomography, magnetic resonance imaging, computed tomography, and/or invasive and non-invasive hemodynamics.

In a further aspect, the nucleotide sequence can code for a brain isoform of creatine kinase. In another aspect, the nucleotide sequence can code for a muscle isoform of creatine kinase. In another aspect, the nucleotide sequence can code for a mitochondrial isoform of creatine kinase. In yet a further aspect, the nucleotide sequence can code for human isoforms of creatine kinase, including a brain isoform, a muscle isoform, and/or a mitochondrial isoform.

Another aspect of this invention relates to novel methods for treating a subject at risk for or with heart failure using a vector construct having a nucleotide sequence coding for creatine kinase so as to improve adverse heart remodeling. A vector construct having a nucleotide sequence for a mammalian creatine kinase is administered to a subject's heart causing an improvement in ventricular size or shape.

In another aspect, the improvement can comprise and/or be determined by a number of measures including but not limited to, the mammal's end diastolic volume, end systolic volume, ventricular wall thickness, and/or ventricular mass.

In a further aspect, the improvement in adverse heart remodeling can be measured by a number of methods including but not limited to, x-ray, x-ray angiography, ultrasound or echocardiography, nuclear medicine ventriculography or scintigraphy, positron emission tomography, magnetic resonance imaging, computed tomography, and/or by the use of biomarkers that correlate with remodeling.

In a further aspect, the nucleotide sequence can code for a brain isoform of creatine kinase. In another aspect, the nucleotide sequence can code for a muscle isoform of creatine kinase. In another aspect, the nucleotide sequence can code for a mitochondrial isoform of creatine kinase. In yet a further aspect, the nucleotide sequence can code for human isoforms of creatine kinase, including a brain isoform, a muscle isoform, and/or a mitochondrial isoform.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 shows magnetic resonance images of a normal subject (a.) and a patient with dilated cardiomyopathy and heart failure (f.) and spatially-localized ³¹P NMR spectra from magnetization transfer studies for the healthy subject (b-e.) and the patient with HF (g-h.). Flux through the creatine kinase reaction can be measured with magnetization transfer techniques that involve use of a chemically-selective magnetization pulse that saturates a particular magnetic resonance of a metabolite of interest. This figure shows that the gamma-phosphate resonance of ATP can be saturated with the chemically-selective magnetization pulse (c, e, h, where the arrow indicates the frequency position of the chemically-selective magnetization pulse at approximately −2.5 ppm relative to PCr). The reduction in the creatine phosphate resonance during gamma-ATP saturation (c, e, h), as compared to spectra collected during control irradiation (b, d, g, where the arrow indicates the frequency position of the chemically-selective magnetization pulse at approximately +2.5 ppm relative to PCr), is directly related to the flux through the creatine kinase reaction. CK metabolism is reduced in the patient with HF as evidenced by lower PCr levels (lower PCr peak) during control irradiation (g) while lower flux through CK as evidenced by less of a change in PCr peak height between control irradiation (g) and gamma-ATP irradiation (h) with a lower slope between PCr peaks.

FIG. 2 shows the cardiac creatine phosphate to ATP ratio (a), creatine phosphate concentration (b), ATP concentration (c), as well as the pseudo-first order rate constant (d) and flux through CK (e) in sixteen healthy subjects (“Normal” bars) and in seventeen patients with dilated cardiomyopathy and HF (“CHF” bars). Thus the products and substrates of the CK reaction and CK flux (i.e. phosphoryl transfer through the CK reaction) are measurable in the human heart and are altered in human heart failure and dilated cardiomyopathy.

FIG. 3 shows Western blots demonstrating increased cardiac CK-B expression 7 days after Ad-CK-B transduction.

FIG. 4a shows a timeline indicating TAC (at 0 weeks), whole-heart transduction (AdV Trnfx) (at 2 weeks), and MRI/MRS and pressure-volume loop (PVL) studies (at 3 weeks).

FIG. 4b shows anatomic and functional findings by in vivo MRI in a normal animal and in animals three weeks after TAC surgery and one week after whole-heart transduction (AdV Trnfx) with either adeno-CMV-beta-galactosidase (beta-Gal, n=5) or adeno-CMV-CK-B (CK-B, n=9). The MRI findings in the animals receiving the construct without CK are markedly abnormal while the findings in those receiving the construct containing CK-B are nearly identical to those of the normal animals which did not undergo TAC. The top panel shows a representative axial ¹H MR image (left) and corresponding cardiac ³¹P NMR spectrum (right) from a normal animal. The middle panel shows a representative axial ¹H MR image (left) and corresponding cardiac ³¹P NMR spectrum (right) from a TAC+βGal animal. The bottom panel shows a representative axial ¹H MR image (left) and corresponding cardiac ³¹P NMR spectrum (right) from a TAC+CK-B animal.

FIG. 5 shows mean cardiac PCr/ATP values in normal, control animals (“Control” bar), a significantly reduced value in TAC+βGal animals (“TAC-Bgal” bar), and a mean value in the TAC+CK-B animals (“TAC-CK” bar) nearly identical to that in the normal, control animals which did not undergo TAC.

FIG. 6 at (A.) shows that CK-B transduction significantly improves MRI indices of contractile function, including end systolic volume (ESV), stroke volume (SV), and ejection fraction (EF); at (B.) shows that LV EF correlated with cardiac PCr/ATP after gene therapy (EF=0.254 (PCr/ATP)+0.0655, r²=0.40, P<0.005); and at (C.) shows that LV mass correlated with cardiac PCr/ATP after gene therapy (LV mass=−73.7 (PCr/ATP)+257.4, r²=0.50, P<0.01). Thus, administration of vector coding for CK-B significantly improves the contractile or pump function of dysfunctional hearts and the degree of functional improvement and the attenuation of adverse remodeling correlate with, or depend on, the degree of CK metabolic improvement, in this instance measured by the cardiac PCr/ATP ratio.

FIG. 7 shows a plasmid map for AAV2/9 CK-M.

FIG. 8 at (A.) shows the experimental scheme for AAV2/9 CK-M experiments; at (B.) compares the impact of AAV-CK-M administration to that of AAV Beta Gal (without CK-M) on ¹H magnetic resonance images; at (C.) compares the same on MRI measures of contractile function; and at (D.) compares the same on remodeling as measured by LV mass and LV/body mass. Thus, administration of vector coding for CK-M before the development of dysfunction significantly attenuates the decline in contractile or pump function and adverse remodeling. In fact despite TAC surgery, pump function, as indexed by ejection fraction (EF), is nearly normal (65%-70%) following CK therapy.

FIG. 9 shows that favorable long term contractile effects of AAV2/9 CK-M transduction persist for up to 9 weeks after TAC.

FIG. 10 at (A.) shows myocyte isolation and staining for the marker gene (beta galactosidase); and at (B.) shows mean transduction efficiency in mice following administration of different vectors and different administration routes. Although arterial administration can easily be accomplished in humans, intravenous (IV) administration (at B., “AAV2/9 IV admin”) is the simplest, safest and least invasive and in this mouse model is associated with significant myocyte transfection.

### DETAILED DESCRIPTION OF THE INVENTION

Throughout this disclosure, applicants refer to texts, patent documents, and other sources of information. One skilled in the art can use the entire contents of any of the cited sources of information to make and use aspects of this invention. Each and every cited source of information is specifically incorporated herein by reference in its entirety. Portions of these sources may be included in this document as allowed or required. However, the meaning of any term or phrase specifically defined or explained in this disclosure shall not be modified by the content of any of the sources.

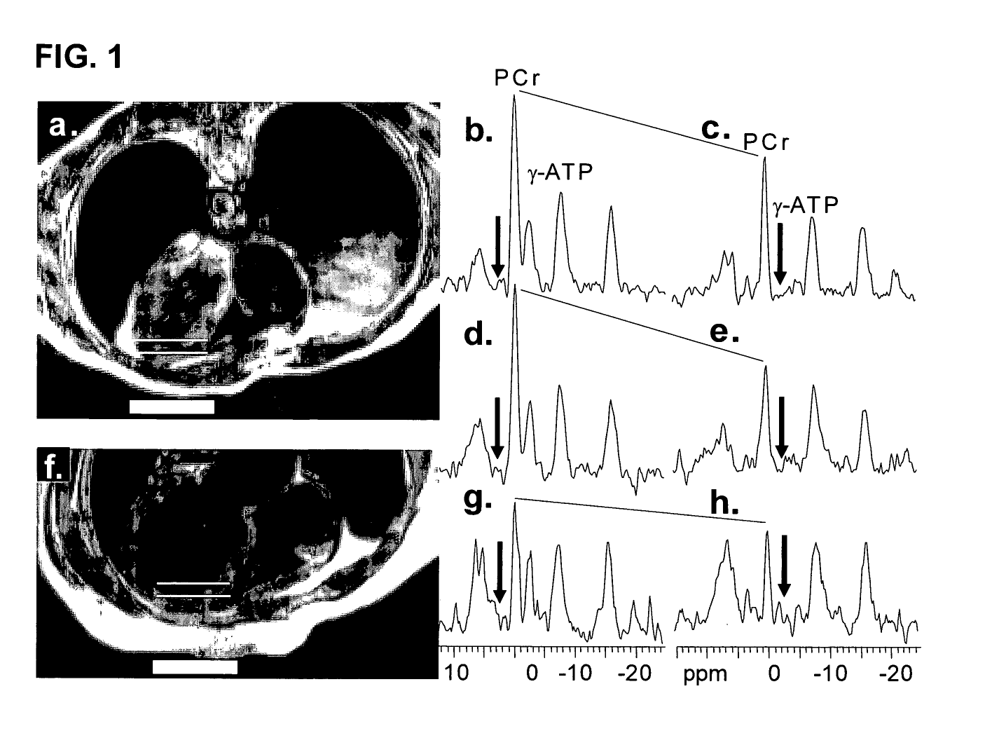

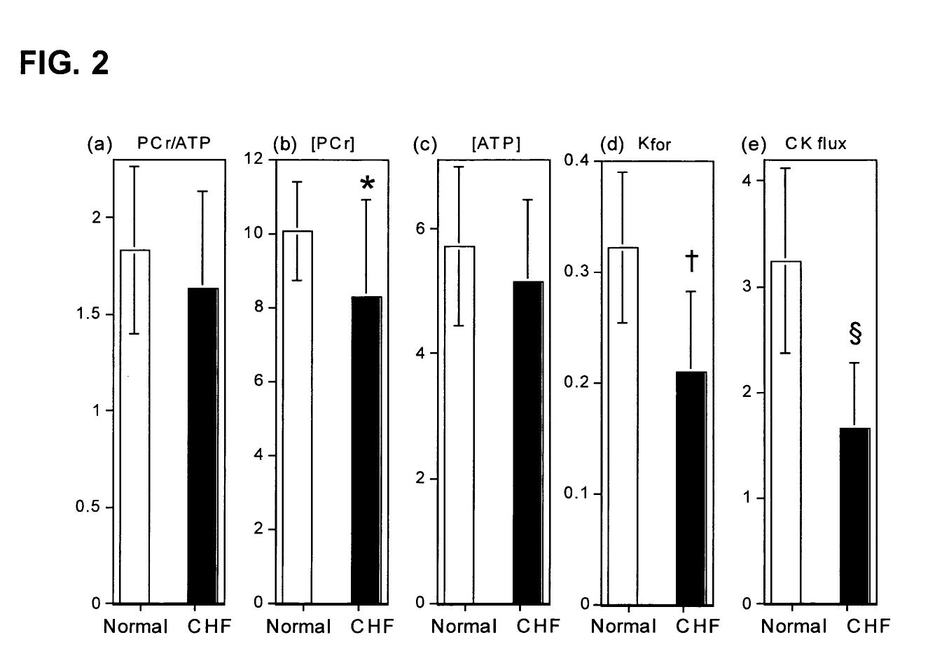

Recent observations in humans offer new insights into the role of reduced CK metabolism in HF. These reveal abnormalities in cardiac CK activity, rather than only reductions in high-energy metabolite pools (i.e. substrates and products), of the CK reaction. ³¹P nuclear magnetic resonance spectroscopy (³¹P NMR spectroscopy) techniques have demonstrated a reduced ratio of creatine phosphate (PCr) to ATP, or the cardiac PCr/ATP ratio, in many forms of human heart failure and this ratio is a strong predictor of overall cardiovascular mortality (Hardy et al. 1991, Neubauer et al. 1997). Recently implemented ³¹P NMR spectroscopy magnetization transfer methods now enable the measurement of flux through the CK reaction in the human heart and demonstrate significant 50%-70% reductions in the rate of CK flux, even before any loss of ATP in HF. These observations were made in two different types of HF, dilated cardiomyopathy (see FIGS. 1 and 2) (Weiss et al. 2005) and hypertrophic cardiomyopathy (Smith et al. 2006). These data show that abnormalities in CK flux are prominent and occur in the most common forms of human HF. However, a means to increase CK activity in normal or failing mammalian hearts was not previously demonstrated. In fact a recent review of strategies to improve energy metabolism in the failing heart in The New England Journal of Medicine does not discuss this option or identify the CK enzyme as a target for augmentation therapy (Neubauer 2007).

Gene therapy and small molecules offer theoretical approaches to augment the activity of a specific enzyme or reaction in cells. However prior approaches have not relied on or exploited either of these to prevent a decrease or to augment reduced CK activity, CK flux, or CK metabolites in heart tissue as a means to treat HF.

In HF the expression and activity of the CK isoforms differ. CK-M and CK-mito are reduced and CK-B is increased in HF (Neubauer et al 1995, Nascimben et al 1996). There is also evidence that the different isoforms may have different affinities for the substrates of the CK reaction. Thus, if someone skilled in the art were to attempt gene therapy or small molecule approaches to improve CK expression or activity in the failing heart, it is unclear which CK isoform should be the target of choice and whether increasing or decreasing CK-B expression, for example, would be of benefit.

The present invention relates to novel methods of increasing, or preventing a decrease in, measures of cardiac creatine kinase metabolism, contractile function, and/or remodeling associated with heart failure. The use of the term “altered,” in the context of creatine kinase expression, is intended to include, but is not limited to, maintaining levels in the context of preventing heart failure or treating mammals at risk for heart failure and indicates an alteration or change as compared to the lower levels that would be expected in a mammal with heart failure.

The use of the terms “increasing” or “increase” throughout this disclosure, including the claims herein, refer to either an actual increase in, or prevention or amelioration of decline in, any of a number of conditions targeted by the novel methods disclosed herein (for example, creatine kinase metabolism, expression, activity, and/or cardiac contractile function). Thus, all aspects of the invention are applicable to treating and preventing (also referred to as treating a mammal at risk for) heart failure. In one aspect, the invention relates to a novel method of increasing CK metabolism in heart cells to improve heart cell function in heart failure. The inventive methods can include increasing or preventing a decline in CK gene expression, CK activity, CK metabolites and/or the transfer of high-energy phosphoryl groups through CK in heart muscle and these can be accomplished by gene therapy or by a pharmacologic agent. In one aspect the invention relates to a method to augment CK metabolism in the heart so as to improve the heart's contractile or pump function as well as to limit the adverse cardiac enlargement (“adverse anatomic remodeling”) that occurs as HF develops and progresses. In yet another aspect, the invention relates to screening potential compounds or agents to assess the effects of the agents on cardiac CK expression and CK activity in both a treatment and preventive model. Thus, the methods for identifying such an agent include agents that increase CK expression and/or activity, as well as agents that prevent or ameliorate the decline in CK expression and/or activity typically associated with heart failure or exposure to an injurious agent or system that mimics characteristics of heart failure.

I. Definitions

The term “heart failure” as used herein, refers to an inability of the heart to pump sufficient blood to meet the needs of the body at normal filling pressures. This includes acquired as well as inherited heart diseases. Several common causes of heart failure and/or cardiomyopathy include, but are not limited to, coronary artery disease and myocardial infarction, infiltrative diseases, hypertension, diabetes, toxins (e.g. alcohol or adriamycin), viruses, heart valve abnormalities, and unknown (idiopathic) causes. Inherited or congenital heart disease and pulmonary diseases, such as primary or secondary pulmonary hypertension, are other causes of heart failure. Heart failure can also be induced in experimental models by several means, including but not limited to, pacing tachycardia and aortic banding or constriction.

The phrase “cardiac contractile function” as used herein, refers to the function of the heart to contract and thereby pump blood throughout the body. Cardiac contractile function is critical for physiologic homeostasis at rest and during exercise and in fact supports physiologic function of all organs in the body. Methods of detecting cardiac contractile function, include, but are not limited to, clinical symptoms, physical findings, laboratory tests and/or invasive and non-invasive hemodynamic measurements. Other methods of detection, include but are not limited to, imaging tests; including but not limited to, x-ray, x-ray angiography, ultrasound or echocardiography, nuclear medicine ventriculography or scintigraphy, positron emission tomography (PET), magnetic resonance imaging (MRI), and/or computed tomography (CT). There are many measures or metrics used to quantify cardiac contractile function in research settings. In clinical practice, the most common measures of contractile function include ejection fraction (the fractional change in the right or left ventricular volume during contraction), stroke volume (the volume of blood ejected with each heart beat) and the cardiac index (the volume of blood ejected per unit of time, normalized for body mass index).

The phrase “cardiac contractile dysfunction” as used herein, means a reduction or impairment in the contraction of the heart or relaxation following contraction. Methods of detecting cardiac contractile dysfunction, include, but are not limited to, the same methods described to assess “cardiac contractile function” and those methods are incorporated herein as examples.

The phrase “adverse heart remodeling” as used herein, means the process of changes in ventricular size or shape. Changes in ventricular size or shape can include, but are not limited to, ventricular cavity dilatation or enlargement and/or heart muscle wall thinning that can occur in diseases of the heart including heart failure and/or increased ventricular mass (e.g. hypertrophy) which is sometimes associated with abnormalities in diastolic relaxation. Clinical measures of adverse heart remodeling are commonly obtained with, but are not limited to, the imaging techniques described above for contractile function and are derived from measures that typically include end diastolic volume (right or left ventricular volume at the end of the relaxation period during a heart beat cycle), end systolic volume (right or left ventricular volume at the end of the systolic contraction during each heart cycle), mass (weight of the heart or heart chamber), heart wall thickness, and tau (time of relaxation after contraction).

The phrase “creatine kinase metabolism” as used herein, means expression of any of the creatine kinase isoforms (including, without limitation, the brain isoform (CK-B), the muscle isoform (CK-M), or the mitochondrial isoform (CK-mito)), and/or activity of the creatine kinase enzyme, and/or flux through the creatine kinase reaction, and/or phosphoryl transfer between creatine phosphate and ATP through the creatine kinase reaction, and/or the pool sizes of the products and/or reactants of the creatine kinase reaction including creatine phosphate, creatine, adenosine triphosphate (ATP) and adenosine diphosphate (ADP).

The phrases “gene delivery” and “gene transfer” as used herein, refer to methods or systems for reliably inserting foreign DNA into target cells, such as into muscle cells. Such methods can result in transient or long term expression of genes. Gene transfer provides a unique approach for the treatment of acquired and inherited diseases. A number of systems have been developed for gene transfer into mammalian cells. See, e.g., U.S. Pat. No. 5,399,346.

The term “DNA” as used herein, refers to a polymeric form of deoxyribonucleotides (adenine, guanine, thymine, or cytosine) in double-stranded or single-stranded form, either relaxed or supercoiled. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. The term captures molecules that include any of the four bases adenine, guanine, thymine, or cytosine, as well as molecules that include base analogues which are known in the art.

The terms “gene” or “coding sequence” or a sequence which “encodes” a particular protein, as used herein, refer to a nucleic acid molecule which is transcribed (in the case of DNA) and translated (in the case of mRNA) in vitro or in vivo when placed under the control of appropriate regulatory sequences. The boundaries of the gene are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and even synthetic DNA sequences.

The term “vector” as used herein, refers to any genetic element, such as a plasmid, phage, transposon, cosmid, chromosome, virus, virion, or any other genetic element, which is capable of replication when associated with the proper control elements and which can function to transfer a gene sequence to a cell. Thus, the term includes cloning and expression vehicles, as well as viral vectors.

The terms “expression vector” or “vector construct” as used herein, refer to an entity that combines a vector with additional elements that can enhance the ability of the vector to transfer a gene sequence to a cell and affect protein expression and can include operably linked promoters and/or enhancers.

The terms “promoter” or “promoter region” as used herein, refer to a region of a DNA in front of (upstream of) the point at which transcription begins (i.e., a transcription start site) and is sometimes referred to in the art as an upstream promoter region, a promoter region or a promoter of a generalized eukaryotic RNA Polymerase II transcription unit.

The terms “operably linked” or “operationally linked” as used herein, refer to an arrangement of elements wherein the components so described are configured so as to perform their usual function. Thus, control elements operably linked to a coding sequence are capable of effecting the expression of the coding sequence. The control elements need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

The term “enhancer” as used herein, refers to a discrete transcription regulatory sequence that can provide specificity of time, location and/or expression level for a particular encoding region (e.g., gene) and can increase the level of transcription of a coding sequence in a cell that contains one or more transcription factors that bind to that enhancer.

The term “AAV vector” as used herein, refers to a vector derived from an adeno-associated virus serotype, including but not limited to, AAV-1, AAV-2, AAV-3, AAV-4, AAV-5, AAVX7, and/or AAV2/9. AAV vectors can have one or more of the AAV wild-type genes deleted in whole or part, preferably the rep and/or cap genes, but retain functional flanking ITR sequences. Functional ITR sequences are necessary for the rescue, replication and packaging of the AAV virion. Thus, an AAV vector is defined herein to include at least those sequences required in cis for replication and packaging (e.g., functional ITRs) of the virus. The ITRs need not be the wild-type nucleotide sequences, and may be altered, e.g., by the insertion, deletion or substitution of nucleotides, so long as the sequences provide for functional rescue, replication and packaging.

The term “recombinant virus” as used herein, refers to a virus that has been genetically altered, e.g., by the addition or insertion of a heterologous nucleic acid construct into the particle.

The term “transfection” as used herein, refers to the uptake of foreign DNA by a mammalian cell. A cell has been “transfected” when exogenous DNA has been introduced inside the cell membrane. A number of transfection techniques are known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties, such as a plasmid vector and other nucleic acid molecules, into suitable cells. The term refers to both stable and transient uptake of the genetic material.

The term “transduction” as used herein, refers to the delivery of a DNA molecule to a recipient cell either in vivo or in vitro, via a replication-defective viral vector, such as via a recombinant AAV virion.

The terms “muscle cell” or “tissue” as used herein, refer to a cell or group of cells derived from muscle, including but not limited to cells and tissue derived from skeletal muscle, smooth muscle, and cardiac muscle. The term captures muscle cells both in vitro and in vivo. The term also encompasses both differentiated and nondifferentiated muscle cells, such as myocytes, myotubes, myoblasts, cardiomyocytes and cardiomyoblasts.

The term “control elements” as used herein, refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present provided the selected coding sequence is capable of being replicated, transcribed and translated in an appropriate host cell.

The term “reporter construct” is a gene or coding sequence that encodes for a protein that can be detected by optical, fluorometric, spectrophotometric, densitometric, nuclear magnetic resonance, radioactive, positron emission tomography or other techniques. One example of a reporter construct is the gene or coding sequence that encodes for firefly luciferase that, when exposed to luciferin and magnesium-ATP generates photons that can be detected with optical techniques.

The term “homology” as used herein, refers to the percent of identity between two polynucleotide or two polypeptide moieties. The correspondence between the sequence from one moiety to another can be determined by techniques known in the art. For example, homology can be determined by a direct comparison of the sequence information between two polypeptide molecules by aligning the sequence information and using readily available computer programs. Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when at least about 80%, preferably at least about 90%, and most preferably at least about 95% of the nucleotides or amino acids match over a defined length of the molecules.

The term “mammal” as used herein, refers to any member of the class Mammalia including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be covered.

The terms “pharmacologic agent” or “pharmacologic composition” as used herein, refers to a biologically active substance that is administered to have a pharmaceutical or medicinal effect. The terms include, but are not limited to, expression vectors, vector constructs, DNA coding for specific peptide or enzyme (e.g. creatine kinase), polypeptides, small molecules with molecular weight less than 2000, proteins, antibodies, hormones, organic and inorganic molecules or compositions.

The term “biomarkers” as used herein refers to a blood or tissue measure that acts as an indicator of a biologic state.

II. Gene Therapy

A. Adenovirus Vector

The use of adenovirus as a vector for cell transfection is well known in the art. Adenovirus vector-mediated cell transfection has been reported for various cells, including the heart (Stratford-Perricaudet, et al., 1992, Champion et al., 2004).

An adenovirus vector of the present invention is replication defective via the deletion of the viral early gene region 1 (E1) and/or E3. It is believed that any adenovirus vector can be used in the practice of the present invention. Thus, an adenovirus vector can be one of any of the 42 different known serotypes or subgroups A-F. An adenovirus is engineered to contain a coding DNA sequence for use as a vector. Such a recombinant adenovirus has been described by Gluzman et al., 1982. Individual DNA sequences such as cDNAs that encode a gene product are inserted into the adenovirus to create a vector construct.

In a preferred embodiment, therefore, a coding sequence for human creatine kinase, including CK-M (Gene bank entry NM 001824.2), CK-B (Gene bank entry NM 001823.3), or CK-mito (Gene bank entry NM 001825.1), is introduced or incorporated into an adenovirus at the position from which the E1/E3 coding sequences have been removed. The propagation of the adenovirus is well known in the art. The particular cell line used to propagate the recombinant adenoviruses of the present invention is not critical to the present invention. Recombinant adenovirus vectors can be propagated on, e.g., human 293 cells, or on other cell lines that are permissive for conditional replication-defective adenovirus infection, e.g., those which express adenovirus E1 gene products “in trans” so as to complement the defect in a conditional replication-defective vector. Further, the cells can be propagated either on plastic dishes or in suspension culture, in order to obtain virus stocks thereof.

B. Adeno-Associated Vectors

Adeno-associated virus (AAV) is a replication-deficient parvovirus, the single-stranded DNA genome of which is about 4.7 kb in length including 145 nucleotide inverted terminal repeat (ITRs). The use of AAV vectors is well known in the art. When AAV infects a human cell, the viral genome integrates into chromosome 19 resulting in latent infection of the cell. Production of infectious virus does not occur unless the cell is infected with a helper virus (for example, adenovirus or herpesvirus). In the case of adenovirus, genes E1A, E1B, E2A, E4 and VA provide helper functions. Upon infection with a helper virus, the AAV provirus is rescued and amplified, and both AAV and adenovirus are produced.

AAV possesses unique features that make it attractive as a vector for delivering foreign DNA into cells. AAV infection of cells in culture is noncytopathic, and natural infection of humans and other animals is silent and asymptomatic. Moreover, AAV infects most (if not all) mammalian cells allowing the possibility of targeting many different tissues in vivo. Kotin et al., EMBO J., 11(13): 5071 5078 (1992) reported that the DNA genome of AAV undergoes targeted integration on chromosome 19 upon infection. Replication of the viral DNA is not required for integration, and thus helper virus is not required for integration. The AAV proviral genome is infectious as cloned DNA in plasmids makes construction of recombinant genomes feasible. Furthermore, because the signals directing AAV replication, genome encapsidation and integration are contained within the ITRs of the AAV genome, the internal approximately 4.3 kb of the genome (encoding replication and structural capsid proteins, rep-cap) may thus be replaced with foreign DNA such as a gene cassette containing a promoter, a DNA of interest and a polyadenylation signal. Another significant feature of AAV is that it is an extremely stable and hearty virus. It easily withstands the conditions used to inactivate adenovirus (56° to 65° C.) for several hours, making cold preservation of AAV-based vaccines less critical. Finally, AAV-infected cells are not resistant to superinfection.

Recombinant vectors derived from an adeno-associated virus (AAV) have been used for gene delivery to several tissues, including the heart (Champion et al., 2004). AAV has a wide host range and is able to replicate in cells from any species so long as there is also a successful infection of such cells with a suitable helper virus. Thus, for example, human AAV will replicate in canine cells coinfected with a canine adenovirus. AAV has not been associated with any human or animal disease and does not appear to alter the biological properties of the host cell upon integration. For a review of AAV, see, e.g., Berns and Bohenzky (1987) Advances in Virus Research (Academic Press, Inc.) 32:243-307. The AAV genome is composed of a linear, single-stranded DNA molecule which contains approximately 4681 bases (Berns and Bohenzky, supra). In this case, the AAV virus would encode CK-M, CK-B, or CK-mito. For a detailed description of the AAV genome, see, e.g., Muzyczka, N. (1992) Current Topics in Microbiol. and Immunol. 158:97-129.

C. Lentiviral Vectors and Other Vectors.

Lentiviruses include members of the bovine lentivirus group, equine lentivirus group, feline lentivirus group, ovinecaprine lentivirus group and primate lentivirus group. The development of lentiviral vectors for gene therapy has been reviewed in Klimatcheva et al., 1999, Frontiers in Bioscience 4: 481-496. Lentivirus containing a transgene has an advantage in that recombinant lentivirus can be used to transduce cells of a subject without resulting in significant toxicity or immunogenicity in the subject, and, following transduction, the transgene is expressed. In this case, the lentivirus would encode CK-M, CK-B, or CK-mito. Other vectors may become available in the future that would be capable of cardiac transfection in mammals and in a preferred embodiment would be included as transfection vectors for CK-M, CK-B, or CK-mito.

D. Coding Sequence

A coding sequence can code for any gene product. A coding sequence can comprise introns and exons provided the coding sequence comprises at least one open reading frame for transcription, translation and expression of that polypeptide. Thus, a coding sequence can comprise a gene, a split gene or a cDNA molecule. In the event that the coding sequence comprises a split gene (contains one or more introns), a cell transduced with a DNA molecule containing that split gene must have a means for removing those introns and splicing together the exons in the RNA transcript from that DNA molecule if expression of that gene product is desired. In some of the alternative embodiments of the invention, CK-M, CK-B, or CK-mito can be encoded by a coding sequence of a vector construct.

E. Enhancer-Promoter

A coding sequence of a viral vector construct is preferably operatively linked to an enhancer-promoter other than an adenovirus/adeno-associated virus/lentivirus enhancer-promoter. A promoter is a region of a DNA molecule typically within about 100 nucleotide pairs in front of (upstream of) the point at which transcription begins (i.e., a transcription start site). That region typically contains several types of DNA sequence elements that are located in similar relative positions in different genes. As used herein, the term “promoter” includes what is referred to in the art as an upstream promoter region, a promoter region or a promoter of a generalized eukaryotic RNA Polymerase II transcription unit.

Another type of discrete transcription regulatory sequence element is an enhancer. An enhancer provides specificity of time, location and/or expression level for a particular encoding region (e.g., gene). A major function of an enhancer is to increase the level of transcription of a coding sequence in a cell that contains one or more transcription factors that bind to that enhancer. Unlike a promoter, an enhancer can function when located at variable distances from transcription start sites so long as a promoter is present. Means for operatively linking an enhancer-promoter to a coding sequence are well known in the art.

An enhancer-promoter as used in a vector construct of the present invention can include, but is not limited to, any enhancer-promoter that drives expression in a cardiac muscle cell. In an example set forth hereinafter, the human cytomegalovirus (CMV) immediate/early gene promoter has been used to result in high-level expression of a gene in the cardiac myocardium. However, the use of other viral or mammalian cellular promoters which are well-known in the art is also suitable to achieve expression of the gene product provided that the levels of expression are sufficient to achieve a physiologic effect specifically in the cardiac myocytes. Exemplary and preferred enhancer-promoters are the CMV promoter, the Rous sarcoma virus (RSV) promoter and the muscle-specific creatine kinase (MCK) enhancer (Zambetti et al., 1992; Yi et al., 1991 and Sternberg et al., 1988). Preferred cardiac muscle specific enhancer-promoters are cardiac isoform troponin C (cTNC) promoter (see e.g., Parmacek et al., 1992 and Parmacek et al., 1990) or myosin heavy chain promoter (MHC) or troponin I. Still further, selection of an enhancer-promoter that is regulated in response to a specific physiologic signal can permit inducible gene product expression.

F. Viral Delivery

Generally, virions are introduced into a muscle cell using either in vivo or in vitro transduction techniques. Suitable methods for the delivery and introduction of transduced cells into a subject have been described. For example, cells can be transduced in vitro by combining recombinant virions with cardiac or cardiac progenitor cells. Transduced cells can then be formulated into pharmaceutical compositions, described more fully below, and the composition introduced into the subject by various techniques, such as by intramuscular, intravenous, intra-arterial, subcutaneous, and intraperitoneal injection, or by injection into cardiac muscle or into the pericardial space, using catheter-based or surgical techniques.

For in vivo delivery, the virions will be formulated into pharmaceutical compositions and will generally be administered parenterally, as described above which may include techniques to allow for enhanced cardiac transduction. A novel method for viral transfection to the heart has been described previously and can achieve robust viral transfection using both adenoviruses and adeno-associated viruses (Champion et al. 2004).

Pharmaceutical compositions will comprise sufficient genetic material to produce a therapeutically effective amount of the protein of interest, i.e., an amount of CK-M, CK-B, or CK-mito sufficient to treat heart failure. The pharmaceutical compositions will also contain a pharmaceutically acceptable excipient. Such excipients include any pharmaceutical agent that does not itself induce the production of antibodies harmful to the individual receiving the composition, and which may be administered without undue toxicity. Pharmaceutically acceptable excipients include, but are not limited to, liquids such as water, saline, glycerol and ethanol. Pharmaceutically acceptable salts include, but are not limited to, mineral acid salts such as hydrochlorides, hydrobromides, phosphates, sulfates, and the like; and the salts of organic acids such as acetates, propionates, malonates, benzoates. Additionally, auxiliary substances, such as wetting or emulsifying agents, pH buffering substances, and the like, may be present in such vehicles. A thorough discussion of pharmaceutically acceptable excipients is available in REMINGTON'S PHARMACEUTICAL SCIENCES (Mack Pub. Co., N.J. 1991).

Appropriate doses will depend on the mammal being treated (e.g., human or nonhuman primate or other mammal), age and general condition of the subject to be treated, the severity of the condition being treated, the particular therapeutic protein in question, and its mode of administration, among other factors. An appropriate effective amount can be readily determined by one of ordinary skill in this art.

Thus, a therapeutically effective amount will fall in a relatively broad range that can be determined through clinical trials. For example, for in vivo administration by injection directly to skeletal or cardiac muscle, a therapeutically effective dose will be on the order of from about 10⁶ to 10¹⁵ of adenovirus or AAV virions, more preferably 10⁸ to 10¹⁴ of adenovirus or AAV virions. Other effective dosages can be readily established by one of ordinary skill in the art through routine trials establishing dose response curves. Dosage treatment may be a single dose schedule or a multiple dose schedule. Moreover, the subject may be administered as many doses as appropriate. One of skill in the art can readily determine an appropriate number of doses.

Administration of viral preparations can be accomplished by multiple methods. Such methods include, but are not limited to, infusing a viral vector construct into a blood vessel that perfuses the heart or injecting a viral vector construct directly into a heart muscle such as a ventricular or atrial wall. In a preferred in vivo embodiment, a catheter is inserted into a blood vessel and advanced into a coronary artery that perfuses a portion of the myocardium. Several techniques can be used to enhance viral transduction. By way of example, it has recently been shown that robust viral transduction can take place in a setting of aortic cross-clamping and whole body cooling that prevents hepatic uptake of virus and provides for significant viral transfection (Champion et al., 2004). Physiological conditions are those necessary for viability of the cardiac muscle cell and include conditions of temperature, pH, osmolality and the like. In a preferred embodiment, body temperature is from about 17° C. to about 50° C., more preferably from about 30° C. to about 40° C. and, even more preferably about 37° C. pH is preferably from a value of about 6.0 to a value of about 8.0, more preferably from a value of about 6.8 to a value of about 7.8 and, most preferably about 7.4. Osmolality is preferably from about 200 milliosmols per liter (mosm/L) to about 400 mosm/L and, more preferably from about 290 mosm/L to about 310 mosm/L. Other physiological conditions needed to sustain cardiac muscle cell viability are well known in the art.

III. Pharmacologic Agent Screen

One embodiment of this invention is to increase cardiac CK metabolism or prevent its decline in HF with a pharmacologic agent. One aspect of this invention is a high-throughput screen (HTS) that can evaluate a library of potential compounds so as to identify agents that can increase CK expression and/or CK activity in heart cells. Compound collections for this HTS include, but are not limited to: 40,000 synthetic compounds from ChemBridge (San Diego, Calif.), 1680 synthetic and natural products from MicroSource Discovery (Gaylordsville, Conn.), 42 000 synthetic compounds from NCI-DTP, and 2056 compounds from TimTec (Neward, Del.).

Such agents can increase cardiac CK metabolism and/or prevent its decline in HF by increasing the expression of genes encoding for specific CK isoforms, by increasing the amount of CK protein produced, and/or by increasing the activity of the CK enzyme. mRNA is important for gene expression, central to many functions of the cell, contains complex secondary and tertiary structural folds, and lacks a cellular repair mechanism. The ability of small molecules to interact with RNA is well recognized and these interactions have the potential to prevent or enhance gene expression, achieve allele-specific modulation of gene expression (when allelic sequence differences result in altered RNA conformations), achieve isoform-specific modulation of gene expression and exhibit allosteric effects (Novac et al. Nucleic Acids 2004).

In one embodiment, this invention relates to a method to identify pharmacologic agents that increase the expression of specific genes encoding for one or more of the CK isoforms (CK-M, CK-B, or CK-mito). This would include the steps of isolating cells that normally express at least one isoform of creatine kinase, such as rat neonatal cardiomyocytes or H9c2 cells derived from embryonic or neonatal rat hearts. Myocytes can also be isolated from normal adult mammalian hearts and from others with experimental or clinical HF. Heart failure models in larger animals (e.g. pigs and dogs) are often preferred since they provide larger numbers of cardiac myocytes. Heart failure can be induced by a number of methods that include, but are not limited to, pacing tachycardia (O'Rourke et al. 1999, Wei et al. 2007) and aortic banding (Ye et al. 2001). Myocytes can be isolated from experimental models of heart failure by excising the heart, isolating a region perfused by a single coronary artery, such as the left anterior descending, and perfusing that region with a cold nominally calcium-free solution containing collagenase and protease or other agents to dissociate the cells. Calcium is reintroduced to the perfusion solution and regions of the midventricular wall are dissected free, the cells are disaggregated, filtered through a mesh and stored for use (O'Rourke et al. 1999, Wei et al. 2007). Cardiac myocytes can also be isolated from the human heart for study (del Monte et al. 1995). Thus cells from established lines of normal or failing hearts can be obtained for study. The particular cell line used, the particular culture conditions, isolation technique, and the exact concentrations are not critical to the present invention and one of ordinary skill in the art can recognize that variations can be applied without departing from the concept, spirit, and scope of the invention.

Cells at a concentration of 50,000 cells per ml are transduced in batch with a reporter construct (20 pg per cell) encoding firefly luciferase under control of the CK-M promoter, for example (Phippard and Manning 2003). Transduced cells are plated on 96-well, 384-well or 1536-well plates (Packard, Biotrend Chemicals) at a density of 5,000 cells per well. A library of small molecule test compounds as described above are then added by using a robotic liquid handling device (e.g. BioMEK FX) and the effect of putative agents on CK gene transcription can be determined by using a dual-luciferase reporter assay system (such as that by Promega). The specific number of plates, supplier of the plates, robotic handling device and luciferase reporter system are provided as examples only and are not intended to be limiting. One of ordinary skill in the art can recognize that variations can be applied without departing from the concept, spirit, and scope of the invention.

In another embodiment, the muscle cells are cardiac cells obtained from a mammal with heart failure.

In another embodiment of this invention, one or more of the cells (whether transduced or not) can be exposed to an injurious agent or system that mimics characteristics of heart failure (including, but not limited to, a reactive oxygen species generating system such as exposure to hydrogen peroxide). Some of the cells can then be contacted with any of the small molecule test compounds identified in a library of test compounds and the CK expression of those cells can be compared to that in cells contacted with inactive (control) agents to identify pharmacologic agents that increase reduced CK expression caused by the injurious agent or system. One of ordinary skill in the art can recognize that variations can be applied without departing from the concept, spirit, and scope of the invention.

In another embodiment of this invention, one or more of the cells (whether transduced or not) can be contacted with any of the small molecule test compounds identified in a library of test compounds before exposure to an injurious agent or system as described above and the CK expression of those cells can be compared to that in cells contacted with inactive (control) agents to identify pharmacologic agents (from the small molecule test compounds) that increase CK expression. Increase as used in this embodiment, is meant to refer to the preventing or ameliorating the reduced CK expression that would be expected to result from exposure to the injurious agent or system in the absence of contact with the pharmacologic agent. One of ordinary skill in the art can recognize that variations, including on the comparison made, can be applied without departing from the concept, spirit, and scope of the invention.

Yet another embodiment of this invention is a pharmacologic agent screen for identifying molecules or test compounds that increase CK activity. This is similar to the screen above for mRNA expression but relates to CK activity. Mechanisms that could increase CK activity include, but are not limited to, those which decrease the effects of phosphorylation, methylation (such as O-linked methylation), oxidation, and sulfhydryl inactivation.

The approach to creating a high-throughput screen (HTS) for agents which increase or preserve CK activity involve the general steps of creating a purified source of one of the CK isoforms, placing the CK isoform in a high-throughput system capable of assaying for CK activity, separately contacting each of a series of agents, such as those in a small molecule library of test compounds, with the CK isoform in the HTS system, and then reading the effect of each small molecule on the output reflecting CK activity as it relates to appropriate control and standard compounds and/or to no compound.

One embodiment of this approach to identify a compound to augment CK activity would utilize several common steps used to identify inhibitors of CK-B for chemotherapeutic applications in oncology (Towler et al. Analytical Biochemistry 2000; 279:96-99). A CK-B expression vector (or vectors for CK-M or CK-mito), such as the EcoRI fragment of the mammalian CK-B expression vector can be cloned in the ecoRI site of PET21a+ for incorporation into commercially purchased E. coli. The insert can be amplified with Pfu DNA polymerase and the resulting fragment appropriately cloned to give an E. Coli expression vector pST085. Expression of soluble CK can be optimized by growing E. coli cells with the vectors followed by a 1-2 hour induction period, with an agent like IPTG. When needed, the cells can be lysed and following a series of steps of centrifugation, column elution, fractionation, recentrifugation, and additional screening the resultant CK enzyme protein can be aliquoted and lyophilized. The precise method for obtaining purified creatine kinase enzyme is not critical to the invention. As used herein, purified is not meant to impose a precise measure of purity. Rather, it is meant to refer to creatine kinase enzyme that is separated from other enzymes present in the cells, especially those utilizing ATP or creatine, since the presence of other enzymes could alter the results of the screening method. A person of ordinary skill in this art can easily modify this approach or develop another approach to produce purified creatine kinase enzyme without departing from the concept, spirit, and scope of this invention.

An HTS can then be run using this purified and concentrated CK isoform and a robotic system (e.g. Tomtec Quadra) in a fashion similar to that previously reported by Towler et al. involving mixing (1) 70 ml of 125 mM Trizma, pH 7.5/0.4 mM ADP/8 mM creatine phosphate (Cr—P), (2) a 20-ml air gap, (3) 70 ml of 125 mM Trizma, pH 7.5/5-10 nM CK-B, (4) a 20-ml air gap, and (5) 20 ml of compound in 50% DMSO (final concentration 12.5 mM) in each of the 96-well, 384-well or 1536-well plates. After adding diacetyl, a color forms that is related to CK activity and this can be read by measuring optical density at 530 nm using a plate reader after a certain period of time. Appropriate positive and negative controls can be incorporated. Many conventional assays for creatine kinase activity exist and a number of those can also be adapted to an HTS for the purpose of identifying activators of creatine kinase. This approach is merely one embodiment for performing this step and it is not intended that the particular steps of this HTS limit the invention. Agents that increase this measure of CK activity would be ideal candidates for further testing in both in vitro and in vivo physiologic and heart failure models as described in the examples below.

In another embodiment of this invention, one or more aliquots of the isolated CK isoforms can be exposed to an injurious agent or system that mimics characteristics of heart failure (including, but not limited to, a reactive oxygen species generating system such as exposure to hydrogen peroxide) while other aliquots of isolated CK isoforms are not exposed (control). The exposed and non-exposed CK enzyme can then be contacted with any of the small molecule test compounds identified in a library of test compounds and compared to identify pharmacologic agents that increase reduced CK activity caused by the injurious agent or system. One of ordinary skill in the art can recognize that variations, including on the comparison made, can be applied without departing from the concept, spirit, and scope of the invention.

In yet another embodiment of this invention, one or more aliquots of the isolated CK isoforms can be contacted with any of the small molecule test compounds identified in the library described above before exposure to an injurious agent or system as described above and the CK activity compared to identify pharmacologic agents that prevent or ameliorate the reduced CK activity that would be expected to result from exposure to the injurious agent or system. One of ordinary skill in the art can recognize that variations, including on the comparison made, can be applied without departing from the concept, spirit, and scope of the invention.

### EXAMPLES

The following examples illustrate the different aspects of the invention. These examples should not be construed as limiting, but rather are included for purposes of illustration. The present invention should be construed as limited only by the claims.

### Example 1

Improving Cardiac Contractile Function with an Adenovirus Vector Construct Containing the Gene for CK-B.

This example describes the use of a viral vector construct to increase CK-B expression, improve energy metabolism, increase contractile function and limit adverse heart remodeling in a mouse model of heart failure.

Adeno-CK-B vector with a CMV promoter is created as previously described (Auricchio et al. 2001). The coding sequence of the mouse CK-B gene is amplified from mouse brain cDNA (CLONTECH) by using the following primers: forward, ATGCCCTTCTCCAACAGCCATAA (SEQ ID NO:1); reverse, TAGCTCTTCGACCGTCATCTTC (SEQ ID NO:2). The PCR product is cloned in the PCR 2.1 vector (Invitrogen, Carlsbad, Calif.) and sequenced. The mouse CK-B sequence is then cloned in the pAd-CMV-link shuttle plasmid (Vector Core, Institute for Human Gene Therapy, Univ. of Pennsylvania; CMV indicates the cytomegalovirus promoter). The pAd-CMV transgene plasmid is used for cotransfection of 293 cells (American Type Culture Collection) with ClaI-digested H5.010CMVEGFP viral backbone containing deletions in map units 1-9 of the E1 region and in the E3 region (sub360) to create adenoviruses by homologous recombination. Recombinant plaques are initially isolated through green/white selection and confirmed by restriction enzyme analysis of DNAs extracted by the Hirt procedure. The positive plaques are purified and grown up in 293 cells for CsCl gradient purification.

To induce heart failure, male C57BL/6 mice (8-12 weeks, Jackson Laboratories, Bar Harbor, Me.) are studied and pressure overload is produced by constricting the transverse aorta (TAC) as previously described (Knowles et al. 2001). Prior studies show that this method of TAC results in dilatation, dysfunction and heart failure after 3 weeks (Takimoto et al. 2005a, Takimoto et al. 2005b). In addition, abnormalities in CK metabolism are also detected by ³¹P NMR spectroscopy at this time (Maslov et al. 2006) which are similar to those observed in human HF (Smith et al. 2006).

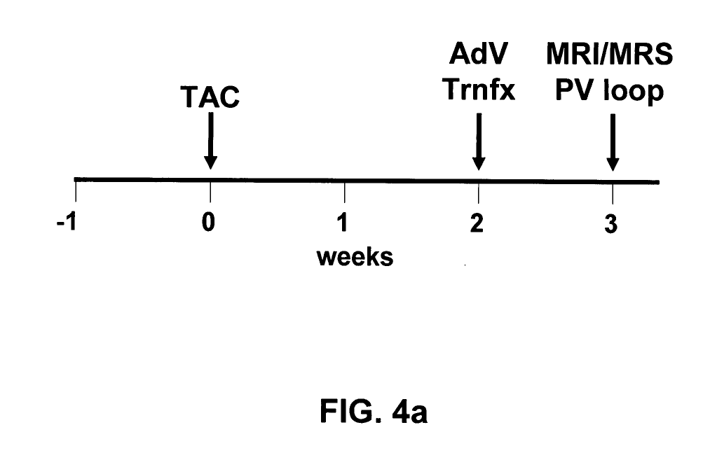

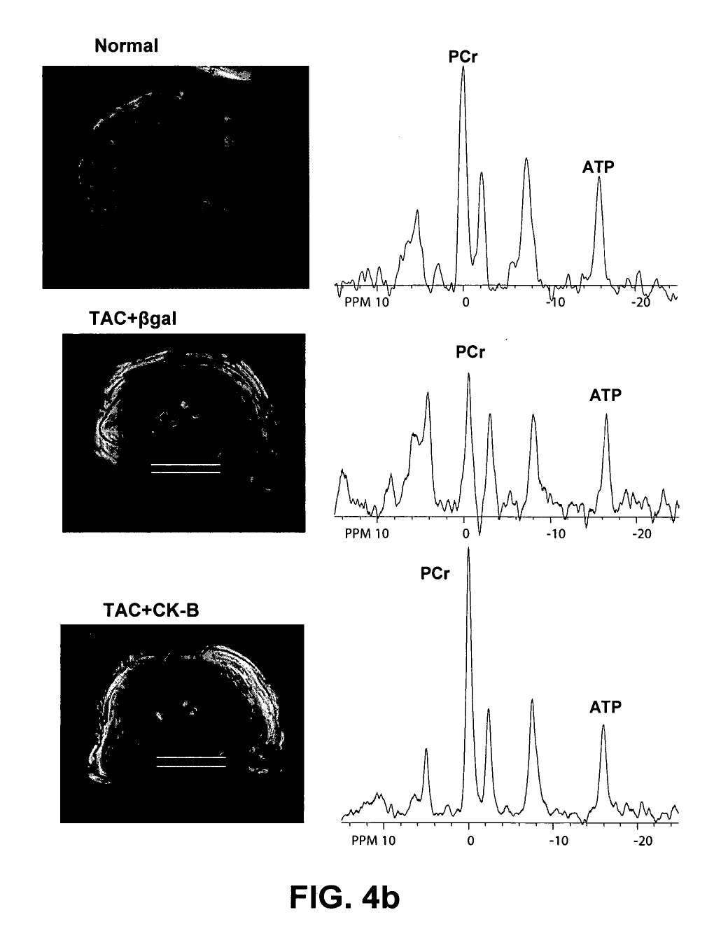

Mice undergo transduction which involves anesthetizing them with isoflurane, cooling them to a core temperature of 19-21° C., clamping the mid-descending aorta, and injecting modified cardioplegic solution into the LV followed by 20 μL of vector as previously described at a dose of 3.3×10¹¹ particles/kg (Champion et al. 2003). The clamp is maintained ford 9 min, then released, and pacing or adrenergic support administered as needed until core temperature rises to 37° C. over 30-40 min (Champion et al. 2003). Because myocardial gene over-expression following adeno-viral transduction peaks at 3-5 days and is sustained at peak levels for an additional 7-10 days (Champion et al. 2003), mice are transduced with the vector construct for adeno-CK-B two weeks after TAC and studied one week after transduction (FIG. 4a).

To determine the in vivo consequences for CK metabolism and function, in vivo MRI/MRS experiments are performed on a Bruker CSI NMR/MRI spectrometer equipped with a 4.7T/40 cm Oxford magnet and actively shielded gradients. The studies are conducted three weeks after TAC, using an in vivo cardiac combined MRI/MRS protocol as previously described (Chacko et al. 2000, Weiss et al. 2002). A complete set of high temporal and spatial resolution multi-slice cine MR images are acquired of the entire LV without gaps to assess mass, ventricular volumes and ejection fraction (Chacko et al. 2000, Weiss et al. 2002). After imaging and without repositioning the animal, a one-dimensional ³¹P chemical shift imaging (1D-CSI) sequence is used to obtain high-energy phosphate data. The PCr and [β-P]ATP peaks in ³¹P NMR localized spectra are quantified by integrating the peak areas (Chacko et al. 2000). In vivo cardiac function is also assessed by the pressure-volume relationship in anesthetized mice using a four-electrode pressure-volume catheter (model SPR-839, Millar Instruments, TX, USA) inserted into the left ventricle via the apex for continuous LV pressure-volume data as previously described (Weiss et al. 2002).

Results are presented as mean ±standard deviation (SD). Comparisons of MRI- and MRS-derived measures of LV anatomy, function and metabolism between two groups (e.g. TAC+βgal vs TAC-CK-B) are performed with the Students t-test while hemodynamic and functional pressure-volume loop (PVL) measures among multiple groups (Table 1) are compared with analysis of variance (ANOVA).

TABLE 1
LV function assessed by pressure volume loop analysis after CK transduction.

| | NORMALS | | AdCMVβgal | | AdCMVCK-B | |
|---|---|---|---|---|---|---|
| | (n = 6) | | (n = 5) | | (n = 9) | |
| | BASELINE | TAC | BASELINE | TAC | BASELINE | TAC |
| HR (min − 1) | 526.4 ± 13.7 | 516.0 ± 18.4 | 521.7 ± 10.6 | 514.7 ± 13.8 | 519.2 ± 9.1 | 512.1 ± 11.7 |
| SBP (mmHg) | 105.0 ± 2.8 | 174.3 ± 5.1* | 101.4 ± 5.7 | 185.2 ± 5.1* | 100.3 ± 5.9 | 182.6 ± 5.2* |
| HW (mg) | 112.6 ± 3.3 | 271.2 ± 7.1* | 112.5 ± 4.4 | 292.1 ± 7.3* | 108.8 ± 3.4 | 235 ± 5.9*# |
| ESV (μL) | 10.2 ± 0.87 | 25.1 ± 3.7* | 11.1 ± 1.8 | 25.8 ± 3.9* | 11.2 ± 1.3 | 17.2 ± 2.3*# |
| EDV (μL) | 30.1 ± 2.5 | 41.2 ± 3.7* | 33.8 ± 6.5 | 41.2 ± 2.4* | 30.3 ± 3.1 | 36.1 ± 2.8# |
| SV (μL) | 20.4 ± 1.9 | 15.9 ± 3.5* | 22.7 ± 2.4 | 15.7 ± 2.1* | 19.7 ± 2.4 | 19.0 ± 2.2# |
| PWRmx/EDV | 29.4 ± 0.6 | 40.2 ± 4.1* | 30.8 ± 2.0 | 35.8 ± 3.7* | 27.0 ± 4.5 | 49.2 ± 5.1*# |
| (mmHg/s) | | | | | | |
| dP/dt max | 13207 ± 495 | 10248 ± 502* | 12230 ± 305 | 10239 ± 386* | 12743 ± 571 | 13030 ± 502# |
| (mmHg/msec) | | | | | | |
| EF (%) | 62.7 ± 2.2 | 42.0 ± 3.6* | 60.7 ± 3.4 | 37.4 ± 4.2* | 67.5 ± 4.2 | 49.0 ± 6.1*# |
| CI (mL/min/m²) | 102.9 ± 9.4 | 78.5 ± 4.0* | 109.7 ± 10.2 | 76.0 ± 6.8* | 104.4 ± 7.0 | 89.6 ± 4.9*# |
| Tau (msec) | 4.0 ± 0.4 | 7.1 ± 0.9* | 4.1 ± 0.3 | 6.7 ± 0.5 | 4.0 ± 0.4 | 5.3 ± 0.3*# |

Abbreviations: HR, heart rate; SBP, systolic blood pressure; HW, heart weight; ESV, end systolic volume; EDV, end diastolic volume; SV, stroke volume; PWRmx/EDV, volume-corrected power index, dP/dtmax, maximum first derivative of pressure over time; EF, ejection fraction; CI, cardiac index; Tau, relaxation constant;
*P < 0.05 vs baseline;
#P < 0.05 vs AdCMVβgal + TAC.

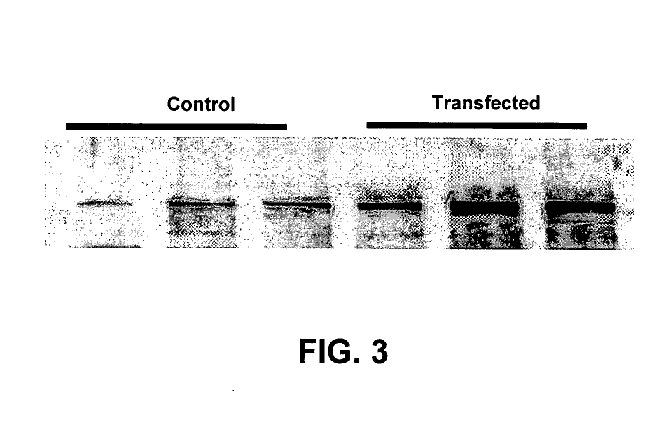

To demonstrate the extent to which this in vivo gene delivery approach can augment CK, normal murine hearts are transduced with an adenovirus coding for CK-B (Ad-CK-B) and this results in a several-fold increase in CK-B expression (FIG. 3), a 40% increase in total CK activity (97±14 vs 68±11 mmol/l/s, P=0.01) and a 140% increase in MB in normal animals. In animals injected two weeks after TAC, Ad-CK-B transduction increases mean total CK activity by 20% (76±8 vs 63±6 mmol/l/s, P=0.02) and increases MB by 180% after 7 days (P=0.004).

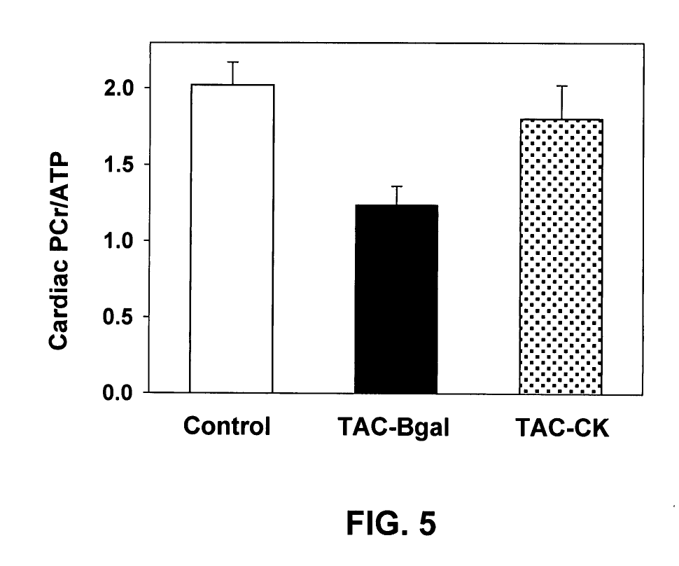

To determine the in vivo metabolic effects of TAC, non-transduced animals are studied after three weeks of TAC when systolic function parameters are decreased. FIG. 4a shows a timeline indicating findings by in vivo MRI three weeks after TAC surgery and one week after whole-heart transduction (AdV Trnfx) with either the control-marker gene adeno-CMV-beta-galactosidase (beta-Gal, n=5) or adeno-CMV-CK-B (CK-B, n=9). FIG. 4b, top panel, shows a representative axial ¹H MR image (left) and corresponding cardiac ³¹P NMR spectrum (right) from a normal animal. FIG. 4b, middle panel, shows a representative axial ¹H MR image (left) and corresponding cardiac ³¹P NMR spectrum (right) from a TAC+βGal animal. FIG. 4b, bottom panel, shows a representative axial ¹H MR image (left) and corresponding cardiac ³¹P NMR spectrum (right) from a TAC+CK-B animal. Mean heart rates during these MRI studies were 602±29 and 588±36 min-1 in the beta-Gal and CK-B hearts, respectively (p=NS). The MRI/MRS findings in the animal receiving the construct without CK are markedly abnormal while the findings in that animal receiving the construct containing CK-B are nearly identical to those of the normal animal. FIG. 5 shows mean cardiac PCr/ATP values in normal animals (“Control” bar), compared to mean cardiac PCr/ATP reduction in TAC+βGal animals (“TAC-Bgal” bar) and mean cardiac PCr/ATP restoration to values in TAC+CK-B animals (“TAC-CK” bar) nearly identical to those in control animals that did not undergo TAC. Thus in vivo mean myocardial PCr/ATP is reduced by 30-40% (FIG. 5), consistent with human heart failure (Conway et al. 1991, Smith et al. 2006). Thus this mouse model of HF reflects PCr/ATP changes of altered cardiac CK metabolism which are similar to those reported in human heart failure.

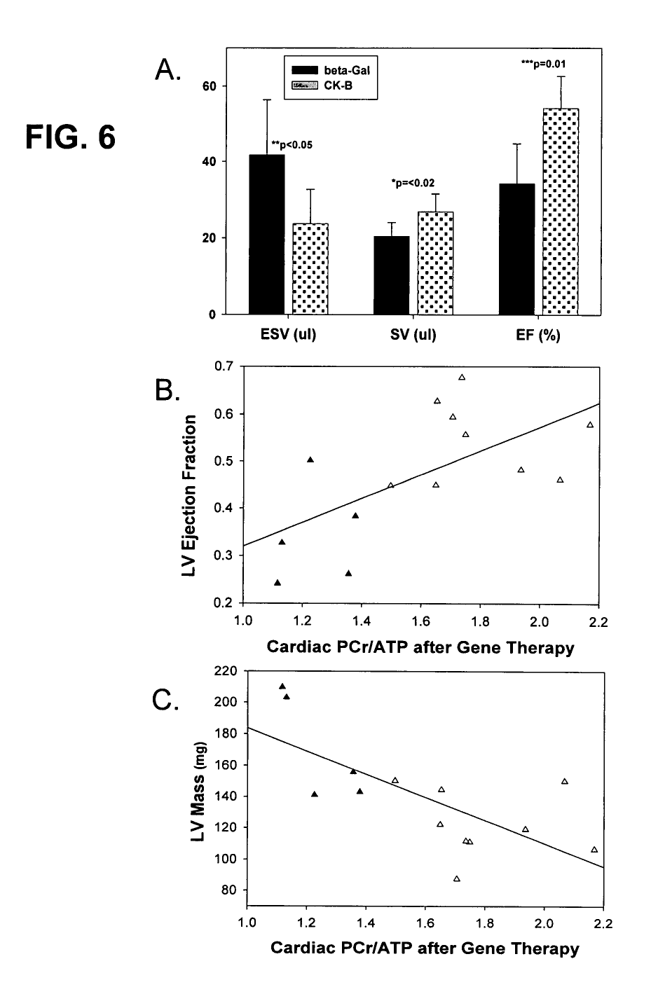

Animals that undergo TAC develop left ventricular hypertrophy, ventricular dilatation, and reduced contractile function (Table 1 and FIG. 6). Table 1 shows findings from catheter-based pressure volume loop studies. In Table 1 it is especially relevant to compare the results of CK-B treated animals after TAC (“AdCMVCK-B”, “TAC” column) with control treated animals after TAC (“AdCMVβGAL”, “TAC” column). In particular, indices of contractile function, including ejection fraction, stroke volume and power index, are significantly better in CK-B treated TAC animals. In addition, indices of adverse anatomic remodeling, including heart weight and end-diastolic volume, are also significantly better in CK-B treated TAC animals. FIG. 6 at (A.) shows that CK-B transduction significantly improves MRI indices of contractile function, including end systolic volume (ESV), stroke volume (SV), and ejection fraction (EF).

Other measures of LV contractile function including dP/dTₘₐₓ and power index, are also significantly better in CK-B transduced TAC animals (Table 1). Cardiac index, a commonly used clinical measure, was significantly better in CK-B treated animals (Table 1, “AdCMVCK-B”, “TAC” column) than in control animals (Table 1, “AdCMVβgal”, “TAC” column). In addition, LV mass by MRI is less in the TAC+CK-B treated than in the TAC+beta gal animals (122±22 mg vs. 171±33 mg, respectively p<0.01) and indices of diastolic relaxation (e.g. tau) are significantly better in the CK-B-treated animals (Table 1). Not only is mechanical function better in TAC+CK-B (FIG. 6; Table 1), but the ejection fraction correlates with cardiac PCr/ATP (FIG. 6 at B.) where EF=0.254 (PCr/ATP)+0.0655, r²=0.40, P<0.005), as does LV mass (LV mass=−73.7 (PCr/ATP)+257.4, r²=0.50, P<0.01) (see FIG. 6 at C.). Thus, gene therapy with CK-B significantly and dramatically improves in vivo metabolism and contractile function as well as limits adverse heart remodeling in failing hearts and the extent of functional and remodeling improvement correlates with the energetic status.

### Example 2

Preventing a Decline in Cardiac Contractile Function by Administering an AAV Vector Construct Containing the Gene for CK-M.

This example describes the use of an adeno-associated virus vector construct to increase CK-M expression, improve energy metabolism, increase contractile function and limit adverse heart remodeling over the course of several months in murine heart failure.

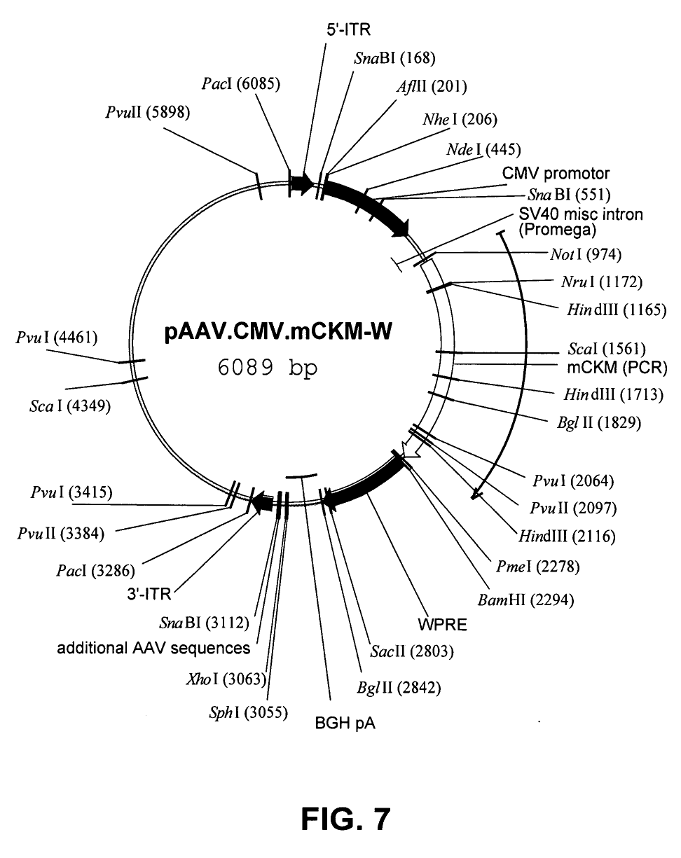

The vector is created as follows. Murine CK-M is cloned from a mouse skeleton muscle cDNA library by PCR. pAAV2-CMV-CK-M is constructed by replacing the EGFP in pAAV2-CMV-EGFP3 with CK-M. The map for this vector appears in FIG. 7. AAV2/9 vectors are produced in 293 cells by triple transfection method using AAV2/9 trans plasmid and purified by three rounds of cesium chloride gradient centrifugation.

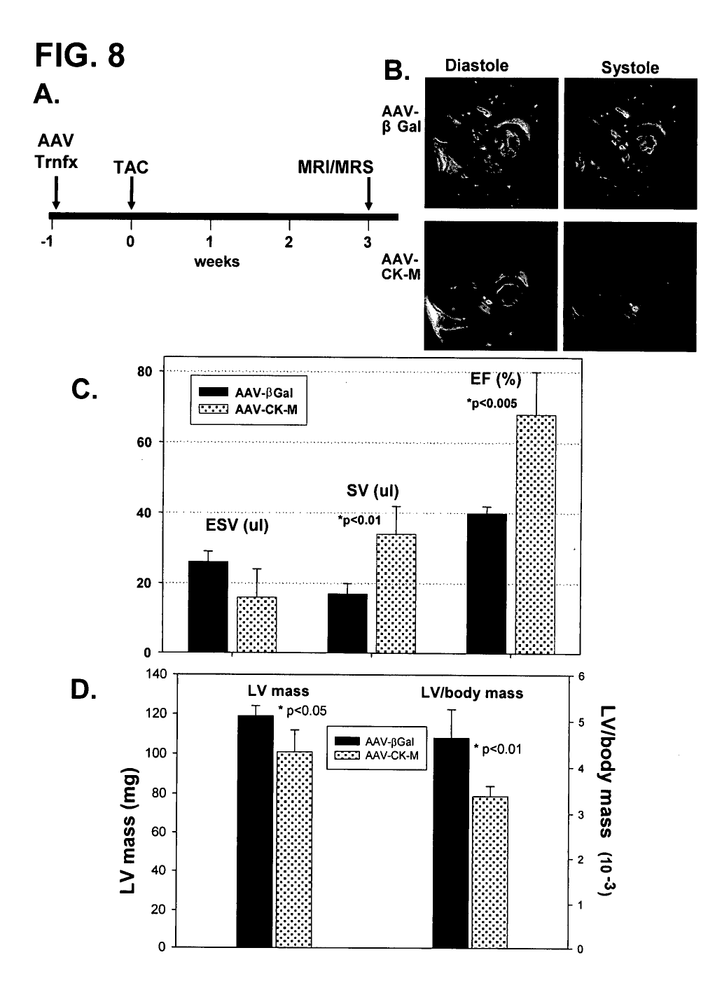

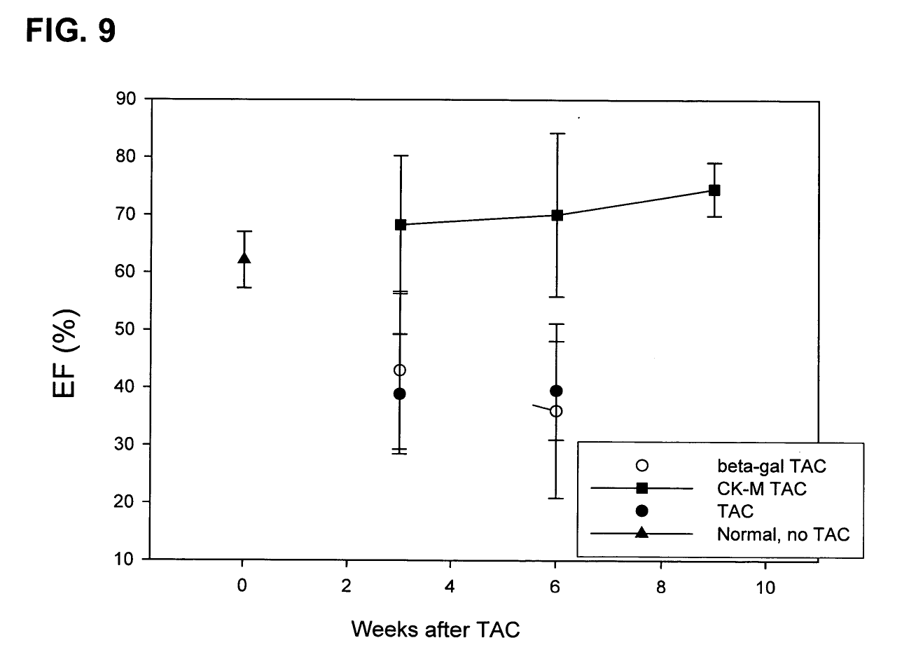

TAC heart failure is induced and this vector is administered as described in Example 1. However, the time of administration precedes TAC by one week (FIG. 8 at A.). In animals transduced with AAV2/9-CK-M, in vivo cardiac PCr/ATP is increased (1.5±0.2 vs 2.5±0.7, TAC+βgal vs TAC+CK-M, respectively, P<0.05) and contractile function is significantly better with higher mean ejection fractions and lower end systolic volumes (FIG. 8 at C.). Specifically, representative end-diastolic and end-systolic LV short-axis MR images obtained three weeks after TAC in animals transduced four weeks earlier with either AAV2/9-βGal or AAV2/9-CK-M (FIG. 8 at A.) demonstrate a smaller LV with better ejection in the CK transduced animals (FIG. 8 at B.). Contractile function is significantly better (FIG. 8 at C.) with higher mean SV and EF in AAV2/9-CK-M (“AAV-CK-M” bars, n=7) than in control AAV2/9-betaGal (“AAV-βGal” bars n=3) transduced mice. In addition the amount of LV hypertrophy is significantly attenuated (FIG. 8 at D.). These results are shown for the same time after TAC as in Example 1. The AAV vector construct allowed long term CK gene expression, and the contractile benefits are observed for up to 9 weeks after TAC (FIG. 9), with mean ejection fractions approaching those of normal animals. Thus the decline in contractile function and the extent of adverse heart remodeling are significantly attenuated following cardiac CK transduction and improvement in CK metabolism, in this instance indexed by improved cardiac PCr/ATP.

### Example 3

Increasing CK Flux in Human Heart Failure.

A process of the current invention can be used to improve contractile function and limit adverse heart remodeling in human heart failure which includes the intravenous administration of a vector including AAV2/9-CK-B.

Patients with heart failure can be identified by one or more clinical findings suggesting compromised contractile function such as symptoms or physical findings of heart failure, by imaging studies demonstrating reduced ventricular ejection fraction, impaired diastolic filling, ventricular dilatation, and/or by chest x-ray findings. Findings of abnormal cardiac creatine kinase metabolites or creatine kinase flux by ³¹P NMR spectroscopy can also be present (FIGS. 1 and 2) and, although not required to make the diagnosis of heart failure, could be used to guide the intervention and document pre-treatment levels or CK flux. The patients can be initially tested for the presence of antibodies directed against the AAV used to prepare the vector construct. If antibodies are present, the patient is preferably given a test dose of from about 10³ to 10⁶ recombinant AAV particles.

Recombinant AAV comprising a coding sequence for one of the CK genes, in this case the one encoding for human CK-B, is prepared and purified by any method that would be acceptable to the Food and Drug Administration for administration to human subjects and proven to have sufficient efficacy and purity for human use.

The AAV-vector construct is administered to patients, preferably by means of intravascular administration in a suitable pharmacologic composition, either as a bolus or as an infusion over a period of time. In this example, the vector can be administered intravenously. This could be given via a peripheral vein or via a central vein using a catheter placed in the jugular vein or in the femoral vein such as commonly performed in cardiac catheterization.

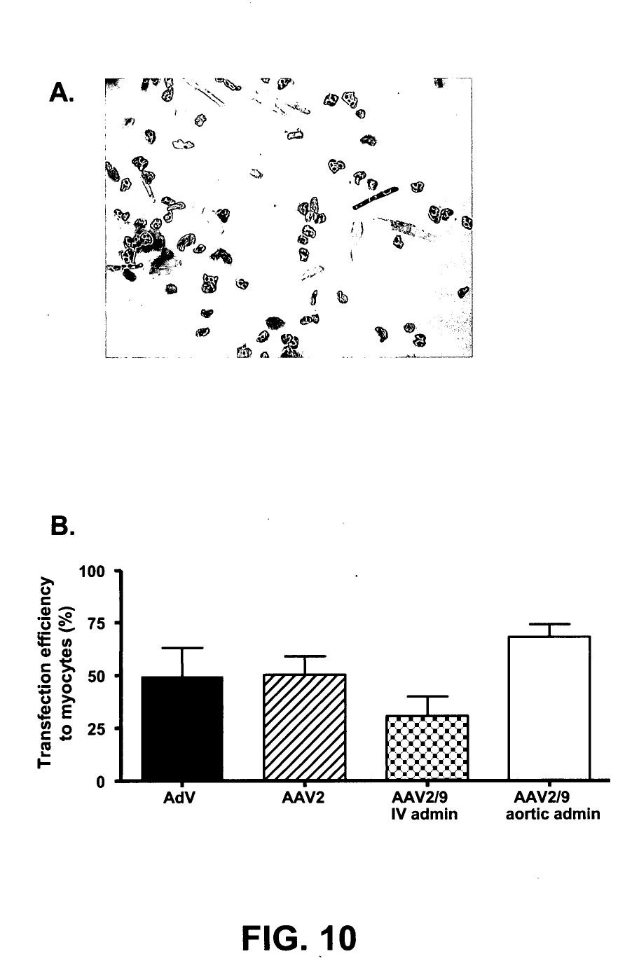

Recent data demonstrate that intravenous administration of AAV2/9 serotype achieves significant cardiac myocyte transduction rates that are comparable to those obtained with the intra-aortic administration of adenovirus that produces contractile benefits in failing hearts (see Example 1 above). Myocytes are isolated from mouse hearts transduced with beta-galactosidase and are stained to identify transduced individual cells, identified in FIG. 10 (at A.) with blue nuclei. Using this approach, the fraction of myocytes transfected is quantified (FIG. 10 at B.) and found to be approximately 50% following aortic administration of adenovirus (“AdV” bar, and identical to that used in Example 1) and adeno-associated virus serotype 2 (FIG. 10 at B., “AAV2” bar), and almost 70% with adeno-associated virus serotype 2/9 (FIG. 10 at B., “AAV2/9” bar, and identical to the vector and administration route used in Example 2). Intravenous administration of AAV2/9 results in transduction of approximately 40% of myocytes (FIG. 10 at B., “AAV2/9” bar). Thus, myocyte transduction rates achievable by intravenous administration of newer AAV serotypes (e.g. AAV2/9) are comparable to those previously achieved by older adeno- and AAV serotypes using the more invasive, intra-arterial route for administration. In this embodiment, the AAV vector is administered in an effective expression-inducing amount, preferably between 10⁶ and 10¹⁵ virus particles or pfu and most preferably between 10⁸ to 5×10¹² virus particles or pfu.

Patients can remain hospitalized during the trial to monitor acute and delayed adverse reactions such as an inflammatory reaction. The effects of this invention on cardiac contractile function can be monitored with echocardiography, magnetic resonance imaging, computed tomography, nuclear medicine ventriculography or scintigraphy, or conventional x-ray angiography to assess indices such as ejection fraction, end systolic and diastolic volumes, ventricular pressure and rate of pressure change, as well as cardiac index. Exercise capacity, such as that measured in a 6 minute walk, or peak oxygen consumption during exercise can also be assessed and is expected to improve in heart failure patients. It is not essential to monitor the effects of this embodiment of the invention on CK expression or CK metabolism; however this can be done, and may offer diagnostic or prognostic clinical management benefit. The effects on cardiac CK expression can be monitored by analyzing tissue obtained at biopsy such as that obtained during transvenous endomyocardial biopsy. The effects of the invention on CK metabolism, CK flux, CK activity and phosphoryl transfer through CK can be monitored by ³¹P NMR spectroscopy as described and demonstrated above (FIGS. 1 and 2).

### REFERENCES

American Heart Association. Heart Disease and Stroke Statistics—2007 Update. Dallas, Tex.: American Heart Association; 2007.
Auricchio A, Zhao R, Wilson J M, Glickson J D (2001), In vivo detection of gene expression in liver by 31P nuclear magnetic resonance spectroscopy employing creatine kinase as a marker gene, Proc Natl Acad Sci 98: 5205-5210.
Chacko V P, Aresta F, Chacko S M, Weiss R G (2000), MRI/MRS assessment of in vivo murine cardiac metabolism, morphology, and function at physiological heart rates, Am J Physiol 279: H2218-H2224.
Champion H C, Georgakopoulos D, Haldar S, Wang L, Wang Y, Kass D A (2003), Robust adenoviral and adeno-associated viral gene transfer to the in vivo murine heart: application to study of phospholamban physiology, Circulation 108: 2790-2797.
Conway M A, Allis J, Ouwerkerk R, Niioka T, Rajagopalan B, Radda G K (1991), Detection of low phosphocreatine to ATP ratio in failing hypertrophied human myocardium by ³¹P magnetic resonance spectroscopy, Lancet 338: 973-976.
del Monte F, O'Gara P, Poole-Wilson P A, Yacoub M, Harding S E. (1995) Cell geometry and contractile abnormalities of myocytes from failing human left ventricle. Cardiovasc Res. 30:281-290.
Hardy C J, Weiss R G, Bottomley P A, Gerstenblith G (1991), Altered myocardial high-energy phosphate metabolites in patients with dilated cardiomyopathy, American Heart Journal 122: 795-801.
Horn M, Remkes H, Dienesch C, Hu K, Ertl G, Neubauer S (1999), Chronic high-dose creatine feeding does not attenuate left ventricular remodeling in rat hearts post-myocardial infarction., Cardiovasc Res 43: 117-124.
Ingwall J S (1993), Is cardiac failure a consequence of decreased energy reserve?, Circulation 87: VII-58-VII-62.
Ingwall J S, Kramer M F, Fifer M A, Lorell B H, Shemin R, Grossman W, Allen P D (1985), The creatine kinase system in normal and diseased human myocardium, N Engl J Med 313: 1050-1054.
Katz A M (1998), Is the failing heart energy depleted?, Cardiology Clinics 16: 633-644.
Knowles J W, Esposito G, Mao L, Hagaman J R, Fox J E, Smithies O, Rockman H, Maeda N (2001), Pressure-independent enhancement of cardiac hypertrophy in natriuretic peptide receptor A-deficient mice, Journal of Clinical Investigation 107: 975-984.
Maslov M, Chacko V P, Smith C J, Stuber M, Moens A, Kass D A, Champion H C, Weiss R G. (2007) Serial in vivo MRI/MRS study of murine cardiac function and metabolism following chronic pressure-overload stress. Am J Physiol 292:H387-391.
Nascimben L, Ingwall J S, Pauletto P, Friedrich J, Gwathmey J K, Saks V A, Pessina A C, Allen P D (1996), Creatine kinase system in failing and nonfailing human myocardium, Circulation 94: 1894-1901.
Neubauer S (2007), The failing heart—an engine out of fuel, N Engl J Med 356: 1140-1151.
Neubauer S, Horn M, Naumann A, Tian R, Hu K, Laser M, Friedrich J, Gaudron P, Schnackerz K, Ingwall J S, Ertl G. (1995), Impairment of energy metabolism in intact residual myocardium of rat hearts with chronic myocardial infarction. J Clin Invest. 1995; 95:1092-1100.
Neubauer S, Horn M, Cramer M, Harre K, Newell J B, Peters W, Pabst T, Ertl G, Hahn D, Ingwall J S, Kochsiek K (1997), Myocardial phosphocreatine-to-ATP ratio is a predictor of mortality in patients with dilated cardiomyopathy, Circulation 96: 2190-2196.
O'Rourke B, Kass D A, Tomaselli G F, Kääb S, Tunin R, Marban E. (1999) Mechanisms of altered excitation-contraction coupling in canine tachycardia-induced heart failure, I: experimental studies Circulation Research 1999; 84; 562-570
Phippard D, Manning A M (2003), Screening for inhibitors of transcription factors using luciferase reporter gene expression in transfected cells, Methods Mol Biol 225: 19-23.
Saks V A (1980), Creatine kinase isozymes and the control of cardiac contraction, in Heart Creatine Kinase, ed. Jacobus W E and Ingwall J S, Williams & Wilkins, Baltimore p 109-124.
Saupe K W, Spindler M, Tian R, Ingwall J S (1998), Impaired cardiac energetics in mice lacking muscle specific isoenzymes of creatine kinase, Circulation Research 82: 898-907.
Smith C S, Bottomley P A, Gerstenblith G, Schulman S P, Weiss R G (2006), Altered creatine kinase ATP kinetics in failing hypertrophied human myocardium, Circulation 114: 1151-1158.
Starling R C, Hammer D F, Altschuld R A (1998), Human myocardial ATP content and in vivo contractile function., Mol Cell Biochem 180: 171-177.
Takimoto E, Champion H C, Belardi D, Moslehi J, Mongillo M, Mergia E, Montrose D C, Isoda T, Aufiero K, Zaccola M, Dostmann W R, Smith C J, Kass D A (2005a), cGMP Catabolism by phosphodiesterase 5A regulates cardiac adrenergic stimulation by NOS3-dependent mechanism, Circulation Research 96: 100-109.
Takimoto E, Champion H C, Li M, Belardi D, Ren S, Rodriguez E R, Gabrielson K, Wang Y, Kass D A (2005b), Chronic inhibition of cyclic GMP phosphodiesterase 5A prevents and reverses cardiac hypertrophy, Nature Medicine 11: 214-222.
Wallimann T (1994), Bioenergetics. Dissecting the role of creatine kinase, Curr Biol 4: 42-46.
Wei S, Ruknudin A M, Shou M, McCurley J M, Hanlon S U, Elgin E, Schulze D H, Haigney M C P; (2007) Muscarinic modulation of the sodium-calcium exchanger in heart failure, Circulation 115:1225-1233.
Weiss R G, Chatham J C, Georgakopoulos D, Wallimann T, Kay L, Walzel B, Wang Y, Kass D A, Gerstenblith G, Chacko V P (2002), An increase in the myocardial PCr/ATP ratio in GLUT4 null mice, FASEB Journal 16: 613-615.
Weiss R G, Gerstenblith G, Bottomley P A (2005), ATP flux through creatine kinase in the normal, stressed, and failing human heart, Proc Natl Acad Sci 102: 808-813.
Ye Y, Wang C, Zhang J, Cho Y K, Gong G, Murakami Y, Bache R J (2001), Myocardial creatine kinase kinetics and isoform expression in hearts with severe LVH hypertrophy, Am J Physiol 281: H376-H386.

We claim:

1. A method for increasing detectable cardiac creatine kinase metabolism in a mammal, comprising:
providing a vector construct having a nucleotide sequence coding for a mammalian creatine kinase;
administering the vector construct to a mammal's heart causing a detectable increase in cardiac creatine kinase metabolism.

2. The method of claim 1, wherein the detectable increase is one or more of: a change in the ratio of cardiac creatine phosphate to ATP; a change in the cardiac concentration of creatine phosphate or ATP; or a change in the flux through the cardiac creatine kinase reaction.

3. A method for increasing cardiac contractile function in a mammal, comprising:
providing a vector construct having a nucleotide sequence coding for mammalian creatine kinase;
administering the vector construct to a mammal's heart causing a detectable increase in cardiac contractile function.

4. The method of claim 3, wherein the detectable increase is one or more of: an increase in the left or right ventricular ejection fraction; or an increase in cardiac index.

5. The method of claim 3, wherein the increase in cardiac contractile function is measured by one or more of: x-ray; x-ray angiography; ultrasound or echocardiography; nuclear medicine ventriculography or scintigraphy; positron emission tomography; magnetic resonance imaging; computed tomography; or invasive or non-invasive hemodynamics.

6. A method for improving adverse heart remodeling in a mammal, comprising:
providing a vector construct having a nucleotide sequence coding for mammalian creatine kinase;
administering the vector construct to a mammal's heart causing an improvement in ventricular size or shape.

7. The method of claim 6, wherein the improvement is one or more of: end diastolic volume; end systolic volume; ventricular wall thickness; or ventricular mass.

8. The method of claim 6, wherein the improvement in ventricular size or shape is measured by one or more of: x-ray; x-ray angiography; ultrasound or echocardiography; nuclear medicine ventriculography or scintigraphy; positron emission tomography; magnetic resonance imaging; computed tomography; or by the use of biomarkers that correlate with remodeling.

9. The method of claim 1, wherein the nucleotide sequence codes for a brain isoform of mammalian creatine kinase.

10. The method of claim 3, wherein the nucleotide sequence codes for a brain isoform of mammalian creatine kinase.

11. The method of claim 6, wherein the nucleotide sequence codes for a brain isoform of mammalian creatine kinase.

12. The method of claim 1, wherein the nucleotide sequence codes for a muscle isoform of mammalian creatine kinase.

13. The method of claim 3, wherein the nucleotide sequence codes for a muscle isoform of mammalian creatine kinase.

14. The method of claim 6, wherein the nucleotide sequence codes for a muscle isoform of mammalian creatine kinase.

15. The method of claim 1, wherein the nucleotide sequence codes for a mitochondrial isoform of mammalian creatine kinase.

16. The method of claim 3, wherein the nucleotide sequence codes for a mitochondrial isoform of mammalian creatine kinase.

17. The method of claim 6, wherein the nucleotide sequence codes for a mitochondrial isoform of mammalian creatine kinase.

18. The method of claim 1, wherein the mammal is a human.

19. The method of claim 3, wherein the mammal is a human.

20. The method of claim 6, wherein the mammal is a human.

21. A method for identifying a pharmacologic agent that increases creatine kinase expression in muscle cells, comprising:
providing isolated muscle cells in culture;
contacting the pharmacologic agent with one or more of the muscle cells; and
detecting an increase in creatine kinase expression.

22. The method of claim 21, wherein the muscle cells are cardiac cells obtained from a mammal with heart failure.

23. The method of claim 21, wherein the muscle cells are transduced with a reporter construct under the control of a creatine kinase promoter and the detection is by a high-throughput system designed to reflect creatine kinase expression.

24. The method of claim 21, wherein the isolated muscle cells are exposed, before or after contact with the pharmacologic agent, to injurious agents or conditions that mimic characteristics of heart failure.

25. A method for identifying a pharmacologic agent that increases creatine kinase activity, comprising:
providing isolated and purified creatine kinase;
contacting the pharmacologic agent with the purified creatine kinase; and
detecting an increase in creatine kinase activity.

26. The method of claim 25, wherein the detection is by a high-throughput system designed to reflect creatine kinase activity.

27. The method of claim 25, wherein the isolated creatine kinase is exposed to injurious agents or conditions that mimic characteristics of heart failure before or after contact with the pharmacologic agent.

28. A method for treating a mammal at risk for or with heart failure, comprising:
providing a vector construct having a nucleotide sequence coding for a mammalian creatine kinase;
administering the vector construct to the mammal's heart causing a detectable increase in cardiac creatine kinase metabolism.

29. The method of claim 28, wherein the detectable increase is one or more of: a change in the ratio of cardiac creatine phosphate to ATP; a change in the cardiac concentration of creatine phosphate or ATP; or a change in the flux through the cardiac creatine kinase reaction.

30. The method of claim 28, wherein the nucleotide sequence codes for a brain isoform of mammalian creatine kinase.

31. The method of claim 28, wherein the nucleotide sequence codes for a muscle isoform of mammalian creatine kinase.

32. The method of claim 28, wherein the nucleotide sequence codes for a mitochondrial isoform of mammalian creatine kinase.

33. A method for treating a mammal at risk for or with heart failure, comprising:
providing a vector construct having a nucleotide sequence coding for a mammalian creatine kinase;
administering the vector construct to the mammal's heart causing a detectable increase in cardiac contractile function.

34. The method of claim 33, wherein the detectable increase is one or more of: an increase in the left or right ventricular ejection fraction; or an increase in cardiac index.

35. The method of claim 33, wherein the increase in cardiac contractile function is measured by one or more of: x-ray; x-ray angiography; ultrasound or echocardiography; nuclear medicine ventriculography or scintigraphy; positron emission tomography; magnetic resonance imaging; computed tomography; or invasive or non-invasive hemodynamics.

36. The method of claim 33, wherein the nucleotide sequence codes for a brain isoform of mammalian creatine kinase.

37. The method of claim 33, wherein the nucleotide sequence codes for a muscle isoform of mammalian creatine kinase.

38. The method of claim 33, wherein the nucleotide sequence codes for a mitochondrial isoform of mammalian creatine kinase.

39. A method for treating a mammal at risk for or with heart failure, comprising:
providing a vector construct having a nucleotide sequence coding for mammalian creatine kinase;
administering the vector construct to a mammal's heart causing an improvement in ventricular size or shape.

40. The method of claim 39, wherein the improvement is one or more of: end diastolic volume; end systolic volume; ventricular wall thickness; or ventricular mass.

41. The method of claim 39, wherein the improvement in ventricular size or shape is measured by one or more of: x-ray; x-ray angiography; ultrasound or echocardiography; nuclear medicine ventriculography or scintigraphy; positron emission tomography; magnetic resonance imaging; computed tomography; or by the use of biomarkers that correlate with remodeling.

42. The method of claim 39, wherein the nucleotide sequence codes for a brain isoform of mammalian creatine kinase.

43. The method of claim 39, wherein the nucleotide sequence codes for a muscle isoform of mammalian creatine kinase.

44. The method of claim 39, wherein the nucleotide sequence codes for a mitochondrial isoform of mammalian creatine kinase.


SEQUENCE LISTING (2 sequences)

SEQ ID NO: 1 - DNA, 23 bp, Murinae gen. sp.
atgcccttctccaacagccataa

SEQ ID NO: 2 - DNA, 22 bp, Murinae gen. sp.
tagctcttcgaccgtcatcttc